(19) United States
(12) Patent Application Publication
(10) Pub. No.: US 2026/0331267 A1
(43) Pub. Date: Oct. 22, 2026

(54) SYSTEM

(71) Applicant: SoftBank Group Corp., Tokyo (JP)

(72) Inventor: Akira SUGIHARA, Tokyo (JP)

(73) Assignee: SoftBank Group Corp., Tokyo (JP)

(21) Appl. No.: 19/647,972

(22) Filed: Apr. 15, 2026

(30) Foreign Application Priority Data
Apr. 22, 2025 (JP) 2025-070242

(51) Int. Cl. G16H 40/20; G08B 21/04; G16H 10/60

(52) U.S. Cl. CPC G16H 40/20; G08B 21/0453; G16H 10/60

(57) ABSTRACT

The present invention relates to a comprehensive decision-making support system that, in a situation where decision-making is difficult such as for an elderly person, integrally manages various personal data such as a medical history, lifestyle habits, values, family relationships, legal information, and an advance directive of the person himself/herself, analyzes a situation in real time using generative AI in an emergency or at the time of a situation change, presents an optimal option for medical and nursing care comprehensively considering values and wishes of the person himself/herself, intentions of a family, a situation of a medical site, and social ethical codes, supports decision-making based on consent of the person himself/herself and the family by explaining a basis and a reason for the option with high transparency in a natural language, and further enables a scenario simulation and a dynamic update of the advance directive.

### CROSS-REFERENCE TO RELATED APPLICATION

The present application claims priority to and incorporates by reference the entire contents of Japanese Patent Application No. 2025-070242 filed in Japan on Apr. 22, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

The problem to be solved by the present invention is the problem that, in a small household such as an elderly couple, when the person himself/herself or a spouse faces emergencies such as a sudden illness or an accident, it is difficult to make optimal decision-making for medical and nursing care comprehensively and promptly considering a wide variety of factors such as intentions and values of the person himself/herself, intentions of a family, a situation of a medical site, legal requirements, and social ethical codes, and specifically, it is to comprehensively solve multi-layered and realistic problems such as: the person himself/herself and the family cannot sufficiently understand complex options regarding medical and nursing care and contents of legal procedures, and cannot make appropriate judgments and expressions of intentions; a medical and nursing care policy is determined without the right to self-determination and dignity of the person himself/herself being sufficiently respected; wishes of the person himself/herself are not accurately conveyed when the family lives far away or when communication is difficult; an advance directive and an expression of intentions cannot be flexibly and dynamically updated according to a change in a situation; and furthermore, the family and medical workers are forced to bear a mental and temporal burden in an emergency, making it difficult to form an optimal consensus and share information.

### SUMMARY OF THE INVENTION

Means for solving the problem according to the present invention is to configure a system comprising a personal information management unit, a situation analysis unit, and a decision-making support unit, wherein the personal information management unit has a function of acquiring various personal data such as a medical history, lifestyle habits, values, family relationships, legal information, and an advance directive of a user from an external service or a communication terminal, managing the personal data in an integrated and dynamic manner, and updating the personal data in real time according to a change in a situation; the situation analysis unit is configured to analyze a health condition, behavior data, a communication history of the user, information from a medical site, and the like in an emergency using generative AI, and determine a degree of urgency and a necessary response; and the decision-making support unit is configured to comprehensively consider values and wishes of the user himself/herself, intentions of a family, a situation of the medical site, social ethical codes, and the like based on information obtained from the personal information management unit and the situation analysis unit, propose an optimal option using an ethical decision-making algorithm by the generative AI, further explain a basis and a reason for the option with high transparency in a natural language, and support decision-making based on understanding and consent of the user and the family.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor indicated by reference numeral (hereinafter simply referred to as "processor") may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), among others.

In the following embodiments, a RAM (Random Access Memory) indicated by reference numeral is a memory where information is temporarily stored and used as a work memory by the processor.

In the following embodiments, a storage indicated by reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) indicated by reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, "A and/or B" means "at least one of A and B." In other words, "A and/or B" means it may be only A, only B, or a combination of A and B. Moreover, when expressing three or more items connected by "and/or," the same concept as "A and/or B" applies.

### First Embodiment

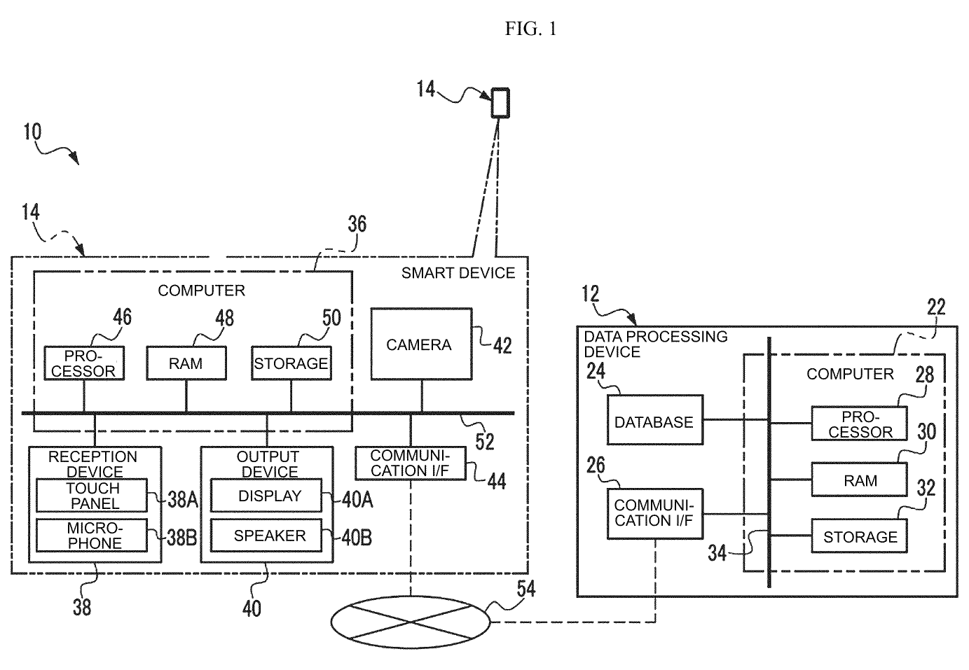

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 is an example of a "computer" related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, camera 42, and communication I/F 44 are also connected to the bus 52.

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A in the processor 46 sends data indicating user input accepted by the touch panel 38A and microphone 38B to the data processing device 12. In the data processing device 12, the specific processing unit 290 acquires data indicating user input.

The output device 40 comprises a display 40A and a speaker 40B, among others, and presents data to the user 20 by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

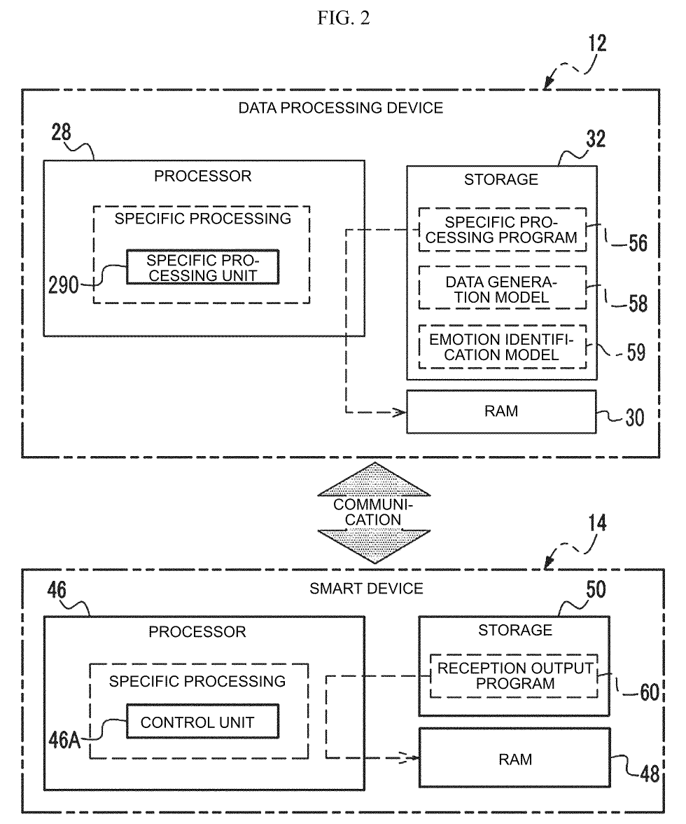

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a "program" related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, and various estimations and predictions related to the user's emotions are performed, but it is not limited to such examples. Furthermore, emotion estimation and prediction may include, for example, emotion analysis.

In the smart device 14, reception output processing is performed by the processor 46. The storage 50 stores a reception output program 60. The reception output program 60 is used in conjunction with the specific processing program 56 by the data processing system 10. The processor 46 reads the reception output program 60 from the storage 50 and executes it on the RAM 48. The reception output processing is realized by the processor 46 operating as a control unit 46A according to the reception output program 60 executed on the RAM 48. The smart device 14 may also have similar data generation models and emotion identification models as the data generation model 58 and emotion identification model 59, and perform the same processing as the specific processing unit 290 using these models. The reception output processing is realized by the processor 46 operating as the control unit 46A according to the reception output program 60 executed on the RAM 48.

Other devices besides the data processing device 12 may have the data generation model 58.

For example, a server device (e.g., a generation server) may have the data generation model 58.

In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

(Example 1)

The flow of the specific processing in Example 1 will be described. Each part of the system described below is realized by the data processing device 12 and the smart device 14. Additionally, the data processing device 12 is referred to as a "server," and the smart device 14 is referred to as a "terminal."

### Embodiment for carrying out the present invention

An embodiment of the present invention is a decision-making support system configured by linking a server and a terminal, and includes a personal information management unit, a situation analysis unit, and a decision-making support unit, wherein each of these units is mainly realized on the server side, and the terminal side is configured to be responsible for an interface with a user and a part of data input/display processing. Hereinafter, the configuration of each unit and a method of utilizing generative AI will be described in detail with comprehensive specific examples. Specifically, the system adopts a distributed computing architecture. The server is composed of a plurality of computing nodes. The computing node includes a parallel computing processor for driving a large language model. The terminal executes a lightweight client application. The system uses edge computing technology together to minimize communication delay between the terminal and the server. The personal information management unit functions as a distributed database system. The situation analysis unit includes a real-time stream processing engine. The decision-making support unit executes a transformer-based neural network. The system containerizes each of these units as a microservice architecture. By this containerization, the system dynamically scales resources according to a load. This configuration significantly improves processing throughput compared to a conventional monolithic system. The system exhibits extremely high availability in a specific use case of real-time decision-making support in a medical site or a nursing care facility. The system does not merely automate a human mental process. The system executes a calculation process peculiar to a computer, which is a semantic similarity calculation in a high-dimensional vector space. This calculation process analyzes complex medical data with a speed and accuracy exceeding human cognitive ability. The system thereby realizes a functional improvement of computer technology itself.

First, the personal information management unit is realized on the server, and manages various personal data such as a medical history, lifestyle habits, values, family relationships, legal information, and an advance directive of a user in an integrated manner. For example, when the user inputs his/her own past illnesses (hypertension, diabetes, heart disease, history of stroke, osteoporosis, presence or absence of dementia, etc.), medication history (antihypertensive drug, antiplatelet drug, insulin, analgesic, etc.), daily health condition (body temperature, blood pressure, pulse, blood glucose level, body weight, meal content, excretion status, etc.), exercise habits (walking frequency, presence or absence of muscle training, rehabilitation record, etc.), dietary habits (salt intake, vegetable intake, drinking frequency, etc.), presence or absence of smoking/drinking, religious beliefs (religious reason for refusing a specific treatment, etc.), wishes for life-prolonging treatment (presence or absence of a wish for cardiopulmonary resuscitation, availability of use of an artificial respirator, presence or absence of a wish for gastrostomy, presence or absence of a wish for palliative care, etc.), family structure and contact information (spouse, child, grandchild, sibling, legal representative, guardian, etc.), legal representative information (adult guardian, voluntary guardianship contract, executor, etc.), and contents of the advance directive (living will, advance medical directive, declaration of death with dignity, designation of a surrogate decision-maker, etc.) from a terminal (smartphone, tablet, personal computer, wearable terminal, etc.), these data are transmitted to the server and accumulated in the personal information management unit. Furthermore, it is also possible to automatically acquire data such as blood pressure, pulse, body temperature, number of steps, sleep time, fall history, hospital visit record, medication alert, rehabilitation progress, and nursing care record (record of excretion, bathing, meal assistance, dementia care, etc.) by API linkage with an external health management service, an electronic medical record system of a medical institution, a nursing care record system, and a wearable terminal (activity meter, electrocardiogram monitor, sleep tracker, fall detection sensor, etc.). The generative AI integrally analyzes these various data, and continuously learns the health condition, decision-making tendency, and change in values of the user. For example, when the user records an improvement in dietary habits or a change in exercise habits, the generative AI automatically detects the change and dynamically updates a profile of values and health risks. Furthermore, information input by proxy from the terminal by a family or a medical worker is also reflected in the personal information management unit, and information serving as a basis for surrogate decision-making is accumulated even when it is difficult for the person himself/herself to express his/her intentions. Specifically, the personal information management unit parses the input various text data by a natural language processing module. The natural language processing module converts the text data into a word embedding vector. The word embedding vector is expressed as, for example, a 768-dimensional floating-point number array. The personal information management unit normalizes time-series health data as a one-dimensional tensor. This normalization process includes interpolation of missing values and removal of outliers. The personal information management unit inputs these heterogeneous data to a multimodal integration layer. The multimodal integration layer learns a correlation between each data modality using a self-attention mechanism. The generative AI is a large language model based on a transformer architecture. Input data to the generative AI is a combined tensor of a text vector indicating the past illnesses of the user, a time-series numerical tensor indicating a blood pressure transition for the latest one week, and an embedding vector indicating the contents of the advance directive. Specific examples of the input data include a categorical variable vector indicating hypertension, a normalized numerical vector indicating a systolic blood pressure of 140, and a semantic vector indicating refusal of life-prolonging treatment. The generative AI processes these input tensors by a multi-layer feedforward neural network. An output of the generative AI is a probability distribution vector indicating a current health risk score and stability of values of the user. Specific examples of the output data include numerical data such as a heart disease risk of 0.85 and a value change probability of 0.12. The system uses this output result as an input for a threshold determination process in the subsequent situation analysis unit. The system activates a profile update module when the value change probability exceeds a predetermined threshold. This learning process optimizes weights of the model by an error backpropagation method using a cross-entropy error function. The system thereby dramatically improves the accuracy of data management. The system brings about a technical effect of reducing latency of data retrieval in a use case of dynamic profiling of medical data.

Next, the situation analysis unit is realized on the server, and analyzes a situation of the user in an emergency or a normal time in real time. For example, when an emergency call such as "the person himself/herself has collapsed and is unconscious", "complaining of chest pain", "has fallen and cannot move", "has difficulty breathing", "fever continues", or "symptoms of dementia have rapidly worsened" is input from the terminal, the situation analysis unit comprehensively analyzes the medical history and lifestyle habits data accumulated in the personal information management unit, the latest health monitoring data, a communication history with a family or a caregiver, information from a medical site, and the like by the generative AI. The generative AI automatically determines a degree of urgency and estimates a risk of myocardial infarction or stroke for a situation such as "an 82-year-old man with a history of hypertension and heart disease has lost consciousness", for example. Furthermore, it simultaneously refers to the values of the user, the contents of the advance directive, contact information of the family, and the like, and determines what kind of response is in line with the intentions of the person himself/herself. For example, if the person himself/herself states in the advance directive that he/she "does not wish for cardiopulmonary resuscitation", it is possible to immediately notify an ambulance crew or a medical institution to the effect that "resuscitation measures are not performed and palliative care is prioritized". The generative AI derives an optimal response policy by referring to past similar cases, medical guidelines, and social ethical codes. For example, when the person himself/herself develops dementia and it is difficult to express his/her intentions, surrogate decision-making is supported by comprehensively considering a past speech history of the person himself/herself, intentions of a family, and instructions of a legal representative. Specifically, the situation analysis unit receives an unstructured text input from the terminal in real time. The situation analysis unit divides the received text into a token sequence by a tokenizer. The situation analysis unit converts this token sequence into a 512-dimensional input tensor. The situation analysis unit inputs this input tensor to a pre-trained recurrent neural network. The recurrent neural network updates a hidden state vector holding time-series context information. An input to the generative AI is a text vector of the emergency call, a binary vector indicating an underlying disease of the user, and a numerical matrix indicating the latest vital signs. Specific examples of the input include an embedding vector of a text complaining of chest pain, a binary vector in which a heart disease flag is on, and numerical data indicating a heart rate of 120. The generative AI weights and evaluates these input data by an attention mechanism. An output of the generative AI is a continuous value score from 0 to 1 indicating the degree of urgency, and a class label probability distribution of a recommended medical treatment. Specific examples of the output include a degree of urgency score of 0.95, a probability of a myocardial infarction response class of 0.88, and a probability of a stroke response class of 0.05. The situation analysis unit determines whether or not this output score exceeds a preset emergency threshold. When determining that the threshold is exceeded, the situation analysis unit immediately activates a subsequent notification routing module. The notification routing module transmits an alert packet to an endpoint of an optimal medical institution. The system executes fine-tuning using past similar case data as teacher data. This fine-tuning fine-tunes parameters of the model using a gradient descent method. The system eliminates a conventional judgment process relying on human intuition. The system classifies the situation objectively and at high speed by an unconventional method of distance calculation of feature vectors in a high-dimensional space. The system exhibits a technical effect of preventing a delay in judgment and improving processing efficiency of the entire system in a use case of triage in emergency medical care.

The decision-making support unit is also mainly realized on the server, but closely cooperates with the interface on the terminal side. The decision-making support unit executes an ethical decision-making algorithm by the generative AI based on an analysis result obtained from the situation analysis unit and data of the personal information management unit, comprehensively considers values and wishes of the user himself/herself, intentions of a family, a situation of a medical site, social ethical codes, and the like, and generates an optimal option. For example, it presents individual and specific options such as "do not wish for resuscitation at the time of cardiopulmonary arrest", "prioritize palliative care without performing life-prolonging treatment", "suspend determination of a treatment policy until a family arrives", "do not wish for attachment of an artificial respirator", "refuse gastrostomy", "give top priority to pain relief", and "refuse blood transfusion for a specific religious reason". The generative AI explains a basis and a reason for these options with high transparency in a natural language. For example, it automatically generates an explanation according to the situation, such as "since it is stated in the advance directive of the person himself/herself that he/she does not wish for life-prolonging treatment, and similar intentions have been confirmed from a past dialogue history with the family, it is optimal to respect a natural course without performing cardiopulmonary resuscitation", "since the person himself/herself refuses blood transfusion for a religious reason, it is necessary to consider an alternative treatment", and "since the family is far away, it is recommended to temporarily suspend the determination of the treatment policy and contact the family". Specifically, the decision-making support unit acquires the probability distribution vector output from the situation analysis unit and the value vector extracted from the personal information management unit. The decision-making support unit combines these vectors and maps them to a high-dimensional feature space. The decision-making support unit inputs this combined vector to a transformer-based generative model. The generative model has a network structure of an encoder and a decoder. An input to the generative model is a numerical tensor of a degree of urgency score, a text embedding vector of the advance directive, and a categorical vector indicating the intentions of the family. Specific examples of the input include a tensor with a degree of urgency of 0.95, a 512-dimensional vector indicating refusal of life-prolonging treatment, and flag data indicating consent of the family. The generative model generates tokens autoregressively in a decoder layer. An output of the generative model is a text sequence of recommended options and an array of reliability scores for each option. Specific examples of the output include text data to the effect of foregoing cardiopulmonary resuscitation, its reliability score of 0.92, and text data of palliative care as an alternative. The decision-making support unit inputs this output text sequence to a natural language processing filter. The natural language processing filter verifies grammatical consistency of the generated text and compliance with ethical constraints. The decision-making support unit transmits the text data that has passed this verification to a display module of the terminal. The system optimizes the ethical decision-making algorithm using reinforcement learning. This reinforcement learning defines a reward function compliant with medical guidelines. The system updates weights of the model using a policy gradient method so as to maximize a reward. The system thereby outputs a basis for judgment of AI, which tends to be a black box, as an explicit text. The system brings about a technical effect of ensuring transparency of information presentation and improving display accuracy of a user interface in a use case of decision-making support in end-of-life care.

In addition, the decision-making support unit performs two-way communication with the user himself/herself and the family via the natural language dialogue interface on the terminal side. For example, when the user inputs a question such as "what happens if I have a stroke?", "how to respond if the family is far away?", "what will be the treatment policy if I develop dementia?", "what kind of preparation is necessary if I want to spend my last moments at home?", or "what kind of procedure is necessary if I wish to enter a nursing care facility?", the generative AI explains options for medical and nursing care and contents of legal procedures in an easy-to-understand manner in response to this, and enables the user and the family to input their own intentions and wishes in a dialogue format. The input contents are immediately reflected in the personal information management unit, and the advance directive and the value profile are dynamically updated. For example, when the user inputs "recently, my thoughts on life-prolonging treatment have changed, so I wish for active treatment from now on", the generative AI analyzes the contents and automatically updates a corresponding part of the advance directive. Specifically, the decision-making support unit implements a dialogue management module. The dialogue management module continuously monitors a natural language input from a user. The dialogue management module analyzes input voice or text data in real time. In the case of a voice input, the system converts it into text data by a voice recognition engine using a hidden Markov model or a deep neural network. An input to the generative AI is an embedding vector of a question text of the user, a context matrix indicating a past dialogue history, and a state vector indicating a current profile state. Specific examples of the input include a text vector asking about a response at the time of a stroke, a dialogue history tensor for the past three times, and a current advance directive flag. The generative AI processes these inputs in a multi-head attention layer and extracts context dependencies. An output of the generative AI is a sequence of a response text and a control command vector instructing a profile update. Specific examples of the output include text data explaining a legal procedure and a control signal for turning on an active treatment wish flag. The decision-making support unit passes this control command vector to a subsequent database update transaction. The database update transaction safely rewrites a record of the personal information management unit while performing exclusive control. The system uses a support vector machine or a convolutional neural network together for intention classification of a dialogue. The system thereby converts an ambiguous input of the user into an accurate system command. The system realizes completely automated dialogue processing without intervention of a human operator. The system exhibits a technical effect of minimizing response latency while saving a communication band in a use case of a medical consultation chatbot.

Furthermore, the decision-making support unit has a function of virtually simulating various emergencies and medical and nursing care scenarios by the generative AI. For example, various scenarios are set, such as "when the person himself/herself falls at home at night and is semi-conscious", "when a spouse develops dementia and it becomes difficult to express intentions", "when a family is staying overseas and cannot be contacted immediately", "when a hospital to be admitted is restricted during an epidemic of a new infectious disease", "when it is necessary to receive medical care at an evacuation shelter when a disaster occurs", "when a mass infection occurs in a nursing care facility", and "when the person himself/herself wishes to spend his/her last moments at home", and a result of decision-making based on the values and wishes of the person himself/herself and an influence thereof are presented in a natural language for each scenario. Through these simulations, the user and the family can prepare and review decision-making while experiencing a plurality of situations in advance, and the advance directive and the personal information are automatically updated based on a simulation result. For example, a detailed simulation is executed for each specific scenario, such as "a treatment course and prognosis when active treatment is desired at the onset of a stroke", "a flow of home care when life-prolonging treatment is refused", "a response at a medical institution until a family arrives", and "a flow of surrogate decision-making when dementia progresses". Specifically, the decision-making support unit includes a probabilistic simulation engine based on a Monte Carlo method. The simulation engine generates a plurality of virtual environment variables using a random number generator. An input to the generative AI is a state vector indicating an initial state of a virtual scenario, a health parameter tensor of the user, and a binary matrix indicating environmental constraints. Specific examples of the input include an event code indicating a night fall, a numerical vector indicating current athletic ability, and an environment flag indicating absence of a family. The generative AI inputs these input data to a state transition model. The state transition model calculates a probability distribution of a next state based on a Markov decision process. An output of the generative AI is a time-series tensor of a state change over time, and a list of recommended actions at each time point. Specific examples of the output include survival probability transition data for each hour after onset, and an action code indicating whether to transport by ambulance or wait at home. The decision-making support unit transmits this output time-series tensor to a visualization module. The visualization module converts the tensor data into coordinate data for graph drawing. The system expands variations of the simulation using a generative adversarial network. The generative adversarial network automatically generates a scenario of an extreme edge case by making a generator and a discriminator compete with each other. The system thereby predicts a complex situation change that cannot be covered by simple rule-based branch processing. The system brings about a technical effect of providing highly accurate future prediction while efficiently utilizing calculation resources in a use case of advance care planning.

In addition, each unit realized on the server side is provided with functions such as encrypted communication, multi-factor authentication, and access authority management from the viewpoint of security and privacy protection, so that an appropriate information disclosure range can be set for each related party such as the user himself/herself, the family, and the medical worker. For example, the medical history and values of the person himself/herself can be viewed only by the person himself/herself and the family, and information is disclosed to a legal representative and a medical worker only within a necessary range. The terminal side is provided with a user interface that can be intuitively operated by the user, and enables the user and the family to use the system anytime and anywhere through voice input, voice output, screen display, a notification function, and the like. For example, customization according to characteristics of the user is also possible, such as providing a voice guide function to a visually impaired user and providing a simplified interface to a user with advanced dementia. Specifically, the server encrypts all communication paths using a transport layer security protocol. The system executes a hybrid encryption algorithm combining a public key cryptosystem and a common key cryptosystem. The system drives a multi-factor authentication module combining biometric authentication data and a one-time password in user authentication. The system expands a role-based access control list on a memory. The system executes a verification process of access authority for each data request in units of microseconds. The terminal includes a display control unit that renders a graphical user interface. The display control unit dynamically adjusts a contrast ratio and a font size according to visual characteristics of the user. The system converts text data into natural voice waveform data using a speech synthesis markup language. The system transfers the converted voice waveform data to an audio output buffer of the terminal. The system executes a layout calculation for limiting the number of operation buttons on a screen and enlarging a pixel size of an icon as a simplified interface for a dementia patient. The system offloads these security processing and interface processing to a hardware accelerator. This offload processing significantly reduces a calculation load of a main processor. The system exhibits a technical effect of maintaining a response speed of the system while reducing a data leakage risk in a use case of secure medical data sharing.

As described above, in the embodiment of the present invention, it is possible to provide a system that realizes comprehensive and flexible decision-making support such as integrated management of personal information, real-time analysis of a situation, ethical decision-making support, explanation/dialogue in a natural language, scenario simulation, and dynamic update of an advance directive with generative AI as an essential element, and maximizes respect for dignity and self-determination rights of the person himself/herself while reducing a burden on an elderly person, a family, and a medical worker. For example, it is possible to cope with various use scenes such that an ambulance crew can immediately confirm intentions of the person himself/herself using a terminal at a site when the person himself/herself suddenly changes, a family can participate in decision-making through a terminal even from a distance, a medical worker can determine a treatment policy while referring to values and past intention expressions of the person himself/herself, and a nursing care facility staff can share a state change of the person himself/herself with a family by inputting a daily care record into the system. Furthermore, the generative AI can continuously learn input contents of the user and a situation change, and evolve individually optimized decision-making support. Specifically, the system integrates an inference pipeline of the generative AI as a core process of the system. The system couples a distributed database, a stream processing engine, and a neural network inference engine with an asynchronous messaging queue. This asynchronous coupling eliminates a processing bottleneck between each module. The system generates a response packet in units of milliseconds in response to a query from a terminal of an ambulance crew. The system establishes a two-way real-time communication session using a WebSocket with a family terminal at a remote location. The system transmits a JSON payload for rendering past intention expression data as a time-series graph to a terminal of a medical worker. The system immediately indexes daily care record data transmitted from a terminal of a nursing care facility staff by stream processing instead of batch processing. The generative AI implements an online learning algorithm. The online learning algorithm sequentially updates weights of the model using a newly input data point. The system attenuates weights of old data by introducing a forgetting factor, and adapts the model to the latest situation change. The system thereby sublimates a static database retrieval system into a dynamic and self-evolving inference system. The system brings about a technical effect of maximizing processing throughput while maintaining data consistency of the entire system in a use case of comprehensive medical and nursing care cooperation.

### System Configuration

The system according to the present embodiment includes a personal information management unit, a situation analysis unit, a decision-making support unit, a simulation unit, an interface unit, a data linkage unit, and a security management unit. The personal information management unit manages various personal data such as a medical history, lifestyle habits, values, family relationships, legal information, and an advance directive of a user in an integrated manner. For example, when the user inputs past illnesses such as hypertension, diabetes, heart disease, and history of stroke from a terminal, this is accumulated in a database on a server, information such as an antihypertensive drug, an antiplatelet drug, insulin, and an analgesic is further recorded as a medication history, body temperature, blood pressure, pulse, blood glucose level, body weight, meal content, excretion status, and the like are collected as a daily health condition, walking frequency, presence or absence of muscle training, a rehabilitation record, and the like are stored as exercise habits, and dietary habits, presence or absence of smoking/drinking, religious beliefs, wishes for life-prolonging treatment, family structure and contact information, legal representative information, contents of the advance directive, and the like are also comprehensively managed. Furthermore, it automatically acquires data such as blood pressure, pulse, body temperature, number of steps, sleep time, fall history, hospital visit record, medication alert, rehabilitation progress, and nursing care record by API linkage with an external health management service, an electronic medical record system of a medical institution, a nursing care record system, and a wearable terminal, and also reflects information input by proxy by a family or a medical worker, thereby accumulating information serving as a basis for surrogate decision-making even when it is difficult for the person himself/herself to express his/her intentions. Specifically, the system deploys each of these units as an independent containerized microservice on a cloud infrastructure. The personal information management unit adopts a polyglot persistence architecture combining a relational database and a NoSQL database. The personal information management unit stores past illnesses and medication history, which are structured data, in a table format. The personal information management unit stores a text of the advance directive and an image of daily meal contents, which are unstructured data, in a document-oriented database. The data linkage unit exposes a RESTful application programming interface. The data linkage unit receives a payload in a JSON format from an external system. The data linkage unit executes a parser that extracts a necessary data field from this payload. The data linkage unit converts the extracted data into an internal standardized data format. The system writes high-frequency time-series sensor data transmitted from the wearable terminal into a time-series database. The system applies a data compression algorithm in this writing process to save storage capacity. The system records the information input by proxy in a blockchain technology or a tamper-proof log system. This recording process cryptographically guarantees the origin and integrity of the data. The system exhibits a technical effect of ensuring data interoperability between heterogeneous systems and reducing input errors by automating data collection in a use case of integrated management of medical data.

The situation analysis unit analyzes a situation of the user in an emergency or a normal time in real time, and for example, when an emergency call such as the person himself/herself has collapsed and is unconscious, complaining of chest pain, has fallen and cannot move, has difficulty breathing, fever continues, or symptoms of dementia have rapidly worsened is input from the terminal, comprehensively analyzes the medical history, lifestyle habit data, latest health monitoring data, communication history with a family or a caregiver, information from a medical site, and the like accumulated in the personal information management unit using generative AI, automatically determines a degree of urgency and estimates a risk of myocardial infarction or stroke when an elderly man with a history of hypertension and heart disease loses consciousness, immediately notifies an ambulance crew or a medical institution to prioritize palliative care without performing resuscitation measures if it is described in the advance directive that the person himself/herself does not wish for cardiopulmonary resuscitation, and supports surrogate decision-making by comprehensively considering a past speech history of the person himself/herself, intentions of a family, and instructions of a legal representative when the person develops dementia and has difficulty expressing intentions. Specifically, the situation analysis unit adopts an event-driven architecture. The situation analysis unit subscribes to an emergency call event via a message broker. The situation analysis unit immediately activates an inference worker process upon receiving the event. An input to the generative AI is a 512-dimensional tensor obtained by vectorizing a text of the emergency call, and a multidimensional feature matrix of the medical history acquired from the personal information management unit. Specific examples of the input include an embedding vector of a natural language indicating loss of consciousness, and a binary array including a hypertension flag. The generative AI processes these inputs using a multilayer perceptron and a self-attention mechanism. An output of the generative AI is a score of a floating-point number indicating the degree of urgency, and a probability distribution vector of a predicted disease class. Specific examples of the output include a degree of urgency score of 0.99, a myocardial infarction probability of 0.85, and a stroke probability of 0.10. The situation analysis unit applies a softmax function to the output probability distribution vector to identify a disease class with the highest probability. The situation analysis unit collates the identification result with flag data of the advance directive by a logical product operation. The situation analysis unit generates a control flag indicating necessity of resuscitation measures based on the operation result. The situation analysis unit transmits a notification payload including the control flag to an external medical institution system. The system thereby completely eliminates a judgment delay due to human intervention. The system brings about a technical effect of minimizing latency of data processing and maximizing a system response speed in a use case of triage during emergency transport.

The decision-making support unit executes an ethical decision-making algorithm by the generative AI based on an analysis result obtained from the situation analysis unit and data of the personal information management unit, comprehensively considers values and wishes of the user himself/herself, intentions of a family, a situation of a medical site, social ethical codes, and the like to generate an optimal option, presents individual and specific options such as not wishing for resuscitation at the time of cardiopulmonary arrest, prioritizing palliative care without performing life-prolonging treatment, suspending determination of a treatment policy until the family arrives, not wishing for attachment of an artificial respirator, refusing gastrostomy, giving top priority to pain relief, and refusing blood transfusion for specific religious reasons, explains a basis and a reason for these options with high transparency in a natural language, and automatically generates an explanation according to a situation such as it is optimal to respect a natural course without performing cardiopulmonary resuscitation because it is described in the advance directive of the person himself/herself that life-prolonging treatment is not wished for and similar intentions have been confirmed from a past dialogue history with the family, it is necessary to consider alternative treatment because the person himself/herself refuses blood transfusion for religious reasons, and it is recommended to temporarily suspend determination of a treatment policy and contact the family because the family is far away. Specifically, the decision-making support unit executes a rule-based inference engine and a neural network-based generative model in a hybrid manner. The decision-making support unit first filters absolute contraindications by the rule-based engine. The decision-making support unit next activates the generative model. An input to the generative AI is a disease prediction vector passed from the situation analysis unit, an embedding tensor indicating the values of the user, and a text vector of medical guidelines. Specific examples of the input include a status code indicating a cardiopulmonary arrest state, a semantic vector indicating refusal of life-prolonging treatment, and a token sequence of the latest resuscitation guidelines. The generative AI processes these inputs using a decoder architecture of a transformer. An output of the generative AI is structured JSON data indicating a plurality of treatment options, and a text sequence of a natural language explaining the basis for each option. Specific examples of the output include an option ID, an action code indicating transition to palliative care, and a text character string describing the reason thereof. The decision-making support unit parses the output structured data and converts it into a display object interpretable by an interface unit. In learning of the generative model, the system executes supervised learning using a judgment history of a medical specialist as teacher data. The system minimizes a complex objective function combining a cross-entropy error and a reward term of reinforcement learning as a loss function. The system thereby automatically generates an ethically appropriate and logical explanatory text. The system exhibits a technical effect of ensuring accuracy of information processing and fulfilling accountability to a user in a use case of complex medical policy determination.

A simulation unit virtually simulates various emergencies and medical and nursing care scenarios by the generative AI, sets various scenarios such as a case where the person himself/herself falls at home at night and is semi-conscious, a case where a spouse develops dementia and has difficulty expressing intentions, a case where a family is staying overseas and cannot be contacted immediately, a case where a hospitalization destination is restricted during an epidemic of a new infectious disease, a case where it is necessary to receive medical care at an evacuation shelter when a disaster occurs, a case where a mass infection occurs in a nursing care facility, and a case where the person himself/herself wishes to spend his/her last moments at home, presents a result of decision-making based on the values and wishes of the person himself/herself and an influence thereof in a natural language for each scenario, enables the user and the family to prepare and review decision-making while experiencing a plurality of situations in advance through these simulations, and automatically updates the advance directive and the personal information based on a simulation result. Specifically, the simulation unit executes a discrete event simulation algorithm. The simulation unit calculates state transition while virtually advancing a system clock. An input to the generative AI is a parameter matrix defining an initial state, and a noise vector expressing uncertainty of an environment. Specific examples of the input include a numerical tensor indicating physical damage at the time of a night fall, a random variable indicating an arrival delay time of the family, and categorical data indicating an infectious disease epidemic level. The generative AI inputs these inputs to a recurrent neural network, and predicts a state change for each time step. An output of the generative AI is a time-series array of a health condition score of the user at each time step, and a probability tree of events that can occur. Specific examples of the output include a data structure indicating a consciousness level decrease probability of 0.7 after one hour, and an emergency transport probability of 0.9 after three hours. The simulation unit analyzes the output probability tree using a search algorithm, and extracts a response route with the highest expected value. The simulation unit passes the extracted route information to a natural language generation module to generate a scenario explanation text. The system temporarily stores the simulation result in a cache memory, and responds at high speed upon a re-request from the user. The system thereby streamlines future prediction processing requiring enormous calculation resources. The system brings about a technical effect of calculating multidimensional scenario branching at high speed and improving prediction accuracy of the system in a use case of formulating an advance care plan.

An interface unit is mounted on the terminal side, provides a natural language dialogue interface for performing two-way communication with the user himself/herself and the family, and when the user inputs a question such as "what happens if I have a stroke?", "how to respond if the family is far away?", "what will be the treatment policy if I develop dementia?", "what kind of preparation is necessary if I want to spend my last moments at home?", or "what kind of procedure is necessary if I wish to enter a nursing care facility?", the generative AI explains options for medical and nursing care and contents of legal procedures in an easy-to-understand manner in response to this, enables the user and the family to input their own intentions and wishes in a dialogue format, and immediately reflects the input contents in the personal information management unit to dynamically update the advance directive and a values profile. Specifically, the interface unit operates as a front-end web application or native application. The interface unit maintains a constant connection with the decision-making support unit on the server side using an asynchronous communication technology. The interface unit captures a keystroke or voice input of the user with an event listener. An input to the generative AI is a token array of the captured question text of the user, and a context vector linked to a session ID. Specific examples of the input include an embedding vector of a text asking about a treatment policy at the time of onset of dementia, and a state tensor indicating immediately preceding dialogue contents. The generative AI processes these inputs in an inference pipeline of a large language model. An output of the generative AI is character string data of a response text, and a control command for changing a display state of a user interface. Specific examples of the output include text data explaining a legal procedure, and a rendering instruction code for popping up a consent button on a screen. The interface unit receives the control command and dynamically rewrites a document object model. The interface unit packages new wishes and intentions input by the user into a payload in a JSON format, and transmits the payload to the server. The system adopts a design that minimizes a rendering load on the client side in this series of processing. The system exhibits a technical effect of reducing a communication overhead between the server and the terminal while improving user experience in a use case of automation of medical consultation.

A data linkage unit performs API linkage with an external health management service, an electronic medical record system of a medical institution, a nursing care record system, a wearable terminal, and the like, automatically acquires data such as blood pressure, pulse, body temperature, number of steps, sleep time, fall history, hospital visit record, medication alert, rehabilitation progress, and nursing care record, and provides these data to the personal information management unit and the situation analysis unit, thereby realizing real-time and comprehensive information collection. Specifically, the data linkage unit functions as a gateway module supporting a plurality of communication protocols. The data linkage unit periodically transmits a polling request to a RESTful endpoint of an external system via an HTTPS protocol. The data linkage unit adopts a lightweight publish-subscribe model such as an MQTT protocol for data reception from the wearable terminal. The data linkage unit temporarily stores received raw data in a buffer area on a memory. The data linkage unit executes data cleansing processing on the raw data. The data cleansing processing includes complementation of missing values, filtering of abnormal values, and synchronization of time stamps. The data linkage unit maps the cleansed data to structured data based on an internal common schema. The data linkage unit publishes the structured data to a message queue. The personal information management unit and the situation analysis unit asynchronously consume data from the message queue. The system strictly performs schema verification of data in the data linkage unit. The system isolates data violating a schema as an error log. The system thereby prevents invalid data from being mixed into the system. The system brings about a technical effect of realizing automation of a data collection process and homogenization of data quality and improving processing stability of the entire system in a use case of data integration between heterogeneous systems.

A security management unit is responsible for security and privacy protection of the entire system, includes functions such as encrypted communication, multi-factor authentication, access authority management, and audit log management, sets an appropriate information disclosure range for each related party such as the user himself/herself, the family, and a medical worker, makes the medical history and values of the person himself/herself viewable only by the person himself/herself and the family, and discloses information to a legal representative and a medical worker only within a necessary range, thereby realizing strict information management. Specifically, the security management unit is designed based on a principle of a zero trust architecture. The security management unit executes a strict authentication and authorization process for all internal and external access requests. The security management unit uses an AES 256-bit encryption algorithm for encryption of communication data. In multi-factor authentication, the security management unit executes a time-based one-time password algorithm in addition to verification of a password hash. The security management unit implements an attribute-based access control model for access authority management. The security management unit inputs a role of a user, an IP address of an access source, and a confidentiality level of requested data to an evaluation function. The evaluation function outputs a Boolean value indicating access permission or denial. The security management unit controls query execution to a database based on the evaluation result. The security management unit records all access histories and system events in an immutable audit log database. The audit log database detects falsification of a log using a hash chain technology. The system periodically and automatically executes a vulnerability scan module, and monitors an application state of a security patch. The system exhibits a technical effect of minimizing a data leakage risk due to a cyber attack or internal fraud and maintaining availability and integrity of the system in a use case of protection of highly confidential medical information.

Specific examples of a prompt sentence to be read by the generative AI when implementing the present invention include "82-year-old man, past illnesses are hypertension and heart disease, described in the advance directive that the person himself/herself does not wish for life-prolonging treatment, currently unconscious, what kind of response is optimal at the time of emergency transport, explain options and reasons thereof in a natural language in consideration of the values of the person himself/herself and the intentions of the family", "the person himself/herself develops dementia and has difficulty expressing intentions, simulate a flow of surrogate decision-making based on a past speech history, the intentions of the family, and instructions of a legal representative, and explain an optimal response policy and a basis thereof", "the person himself/herself wishes to spend his/her last moments at home, what kind of medical and nursing care system is necessary when the family is far away, specifically explain necessary procedures and preparations", and "what kind of alternative treatment can be considered in an emergency when the person himself/herself refuses blood transfusion for religious reasons, generate an explanatory text to a medical site and the family". Specifically, the system includes a prompt engineering module. The prompt engineering module dynamically embeds structured data extracted from a database into a prompt template of a natural language. An input to the generative AI is text data of the dynamically generated prompt sentence. Specific examples of the input include a text character string in which variables such as age, past illness, flag of the advance directive, and current vital signs are embedded. The system converts the text character string into a token ID array of numerical values by a tokenizer. The system supplies the token ID array to an input layer of a large language model. The generative AI calculates a dependency relationship between respective words in the prompt using a self-attention mechanism. An output of the generative AI is a token probability distribution of an answer text to the prompt. Specific examples of the output include a text sequence explaining an optimal response policy, and a generation probability score thereof. The system searches for a text sequence with the highest probability using a beam search algorithm, and determines a final output text. The system adds an instruction token forcing a specific output format to the end of the prompt. The system thereby controls the output of the generative AI to be a JSON format or a specific markup format that can be reliably interpreted in subsequent parsing processing. The system brings about a technical effect of suppressing hallucination of the generative AI and dramatically improving accuracy and consistency of an output in a use case of automatic generation and optimization of a prompt.

### Implementation Steps

Step 1: Collection and Registration of Personal Information The user or the family inputs the medical history (hypertension, diabetes, heart disease, history of stroke, osteoporosis, presence or absence of dementia, etc.), medication history (antihypertensive drug, antiplatelet drug, insulin, analgesic, etc.), daily health condition (body temperature, blood pressure, pulse, blood glucose level, body weight, meal contents, excretion status, etc.), exercise habits (walking frequency, presence or absence of muscle training, rehabilitation record, etc.), dietary habits (salt intake, vegetable intake, drinking frequency, etc.), presence or absence of smoking/drinking, religious beliefs, wishes for life-prolonging treatment (presence or absence of wishes for cardiopulmonary resuscitation, availability of use of an artificial respirator, presence or absence of wishes for gastrostomy, presence or absence of wishes for palliative care, etc.), family structure and contact information, legal representative information, and contents of the advance directive (living will, advance medical directive, declaration of death with dignity, designation of a surrogate decision-maker, etc.) of the person himself/herself using the terminal, and these data are accumulated in the personal information management unit of the server. Furthermore, API linkage is performed with an external health management service, an electronic medical record system of a medical institution, a nursing care record system, and a wearable terminal to automatically acquire data such as blood pressure, pulse, body temperature, number of steps, sleep time, fall history, hospital visit record, medication alert, rehabilitation progress, and nursing care record, and information input by proxy by the family or a medical worker is also reflected. Specifically, in this step, the terminal renders a web form for data input. The terminal validates data input by the user in real time by a client-side script. The terminal serializes the data that has passed the validation into a JSON object. The terminal transmits the JSON object to the server as a secure HTTPS POST request. A receiving module of the server parses the request, and maps the data to a database schema of the personal information management unit. The server inputs text data to a natural language processing engine, and automatically executes semantic tagging. The server executes an authorization flow using an OAuth protocol in API linkage with an external system. The server acquires an access token, and acquires time-series health data from an external data endpoint by batch processing or stream processing. The server inputs the acquired data in heterogeneous formats to an ETL pipeline. The ETL pipeline executes extraction, transformation, and load processing of data, and stores the data in a storage in a normalized vector format. The system asynchronously executes this series of data collection processes as a background task. The system exhibits a technical effect of efficiently accumulating high-quality data necessary and sufficient for learning of a machine learning model while reducing an input load on a user in a use case of constructing an initial data set.

Step 2: Detection of Situation Change and Emergency Call When an emergency call such as the person himself/herself has collapsed and is unconscious, complaining of chest pain, has fallen and cannot move, has difficulty breathing, fever continues, or symptoms of dementia have rapidly worsened is input from the terminal, or an abnormal value or abnormal behavior is automatically detected from the wearable terminal or the nursing care record system, the situation analysis unit of the server receives this in real time, and immediately determines a degree of urgency and necessity of a response. Specifically, in this step, the terminal or a wearable device continuously samples high-frequency time-series data from a built-in acceleration sensor or gyro sensor. The device executes a lightweight anomaly detection algorithm in an edge computing environment. The device calculates a moving average and variance of sensor data, and monitors deviation from a preset threshold value. When detecting an anomaly, the device immediately generates interrupt processing and generates an emergency call packet. The situation analysis unit of the server receives the emergency call packet with low latency via a dedicated UDP port or WebSocket connection. The situation analysis unit analyzes header information of the received packet, and determines a priority of an event. The situation analysis unit dynamically changes resource allocation of an inference engine based on the priority. For an event with a high degree of urgency, the situation analysis unit inputs an inference task to a highest priority queue of a GPU cluster. In the case of an emergency call by text input, the system calculates a degree of urgency score of a text using a natural language processing model. The system fires a software trigger corresponding to a hardware interrupt when the score exceeds a threshold value. The system brings about a technical effect of shortening latency from data transmission to start of analysis to a millisecond order and maximizing immediate responsiveness of the system to a fatal situation change in a use case of real-time anomaly detection.

Step 3: Situation analysis and decision-making support prompt generation by generative AI. The situation analysis unit comprehensively analyzes the medical history and lifestyle habits data accumulated in the personal information management unit, the latest health monitoring data, the communication history with the family and caregivers, the information from the medical site, and the like by the generative AI. Specific examples of prompt sentences to be read by the generative AI include "82-year-old male, past illnesses are hypertension and heart disease, the person himself/herself states in the advance directive that he/she does not wish for life-prolonging treatment, currently unconscious, what response is optimal at the time of emergency transport, explain options and reasons thereof in a natural language in consideration of the values of the person himself/herself and the intentions of the family", "the person himself/herself develops dementia and has difficulty expressing intentions, simulate a flow of surrogate decision-making based on a past utterance history, the intentions of the family, and instructions of a legal representative, and explain an optimal response policy and a basis thereof", and the like. Specifically, in this step, the situation analysis unit queries the latest profile data of a target user from a database. The situation analysis unit combines this profile data and received emergency event data to construct a multidimensional feature vector. The situation analysis unit inputs this feature vector to a prompt generation module. The prompt generation module drives a template engine and inserts each element of the feature vector into a placeholder of a natural language. An input to the generative AI is text data of this dynamically synthesized prompt. Specific examples of this input include a text character string in which an age, a past illness, contents of the advance directive, and a current emergency situation are structured and described. The system converts this text character string into a sequence of token IDs by a subword tokenizer. The system inputs this sequence to an encoder layer of a transformer model. The encoder layer calculates a contextual dependency relationship between tokens using a multi-head attention mechanism, and generates a high-dimensional hidden state vector. The system completes preparation for passing this hidden state vector to a decoder layer. The system executes preprocessing for filtering unnecessary information and data that becomes noise in this prompt generation process. The system automatically constructs an optimal input representation for maximizing inference accuracy of the generative AI in a use case of context optimization, and exhibits a technical effect of preventing waste of calculation resources.

Step 4: Presentation and explanation of optimal options by generative AI. The generative AI comprehensively considers the values and wishes of the person himself/herself, the intentions of the family, the situation of the medical site, the social ethical codes, and the like, presents individual and specific options such as not wishing for resuscitation at the time of cardiopulmonary arrest, prioritizing palliative care without performing life-prolonging treatment, suspending determination of a treatment policy until the family arrives, not wishing for attachment of an artificial respirator, refusing gastrostomy, giving top priority to pain relief, and refusing blood transfusion for a specific religious reason, and explains the basis and reason for these options with high transparency in a natural language. Specific examples of prompt sentences to be read by the generative AI include "generate an explanatory sentence that it is optimal to respect a natural course without performing cardiopulmonary resuscitation because it is described in the advance directive of the person himself/herself that he/she does not wish for life-prolonging treatment, and similar intentions have been confirmed from a past dialogue history with the family", "generate a sentence explaining to the medical site and the family that it is necessary to consider an alternative treatment because the person himself/herself refuses blood transfusion for a religious reason", and the like. Specifically, in this step, the decoder layer of the generative AI autoregressively generates an output token based on the hidden state vector received from the encoder. The output of the generative AI is a text sequence indicating an optimal response option, and a text sequence explaining a logical basis for the option. Specific examples of this output include a conclusion text of withholding cardiopulmonary resuscitation, and a reason explanation text based on the advance directive and the past dialogue history. The system applies a post-processing module to the generated text sequence. The post-processing module verifies whether the generated text contains a medical contraindication or an ethical violation using a regular expression or a rule-based checker. The system packages the text data that has passed this verification into a response payload in a JSON format. The system transmits this payload to the requesting terminal. A display control unit of the terminal parses this payload and renders the text on a designated component on a graphical user interface. When the user inputs feedback to the presented option, the system saves the input data in a log database as new learning data. The system executes continuous fine-tuning of the model using this log data. The system brings about a technical effect of rapidly and safely presenting a highly advanced inference result in a human-understandable format in a use case of a final output of decision-making, and improving usefulness and reliability of the entire system.

Step 5: Dialogue with user and family and intention reflection. Via an interface unit, the user and the family can confirm the options and explanations presented by the generative AI on the terminal, and input an additional question or intention declaration in a natural language. For example, in response to a question such as "what happens if I have a stroke?", "how to respond if the family is far away?", or "what will be the treatment policy if I develop dementia?", the generative AI explains options for medical and nursing care and contents of legal procedures in an easy-to-understand manner, and enables the user and the family to input their own intentions and wishes in a dialogue format. The input contents are immediately reflected in the personal information management unit, and the advance directive and a values profile are dynamically updated. Specifically, the interface unit executes a natural language processing model. The natural language processing model is a large language model based on a transformer architecture. The interface unit receives an input from the user. The input is a text character string in a natural language. For example, the input is a character string "what happens if I have a stroke?". The interface unit converts the text character string into a word embedding vector. The word embedding vector is, for example, a 768-dimensional numerical array. The large language model receives the word embedding vector as an input. The large language model analyzes a context using a self-attention mechanism. The large language model outputs a probability distribution of a response text. The large language model generates an optimal response text character string based on the probability distribution. For example, the output is a character string "in the case of a stroke, rehabilitation is performed after acute phase treatment". The interface unit outputs the response text character string to a display module. Furthermore, the interface unit converts the input intentions into structured data. The structured data is a hierarchical data structure consisting of key-value pairs. The interface unit transmits the structured data to the personal information management unit. The personal information management unit automatically updates the database using the structured data. Thereby, the system accurately extracts an intention of the dialogue. The system automates the update of the database. As a result, the system improves accuracy of data input. Furthermore, the system dramatically improves processing speed. The present technology is applied to an intention confirmation process of a patient at a medical site.

Step 6: Scenario simulation and advance preparation by generative AI. A simulation unit virtually simulates various emergencies and medical and nursing care scenarios by the generative AI, sets diverse scenarios such as a case where the person himself/herself falls at home at night and is semi-conscious, a case where a spouse develops dementia and has difficulty expressing intentions, a case where the family is staying overseas and cannot be contacted immediately, a case where a hospitalization destination is restricted during an epidemic of a new infectious disease, a case where it is necessary to receive medical care at an evacuation shelter when a disaster occurs, a case where a mass infection occurs in a nursing care facility, and a case where the person himself/herself wishes to spend his/her last moments at home, presents a result of decision-making based on the values and wishes of the person himself/herself and an influence thereof in a natural language for each scenario, and enables the user and the family to prepare and review decision-making while experiencing a plurality of situations in advance through these simulations. Specific examples of prompt sentences to be read by the generative AI include "specifically explain what kind of medical and nursing care system is necessary, and necessary procedures and preparations when the person himself/herself wishes to spend his/her last moments at home and the family is far away", "simulate a treatment course and prognosis when active treatment is desired at the onset of a stroke, and generate a sentence explaining to the family", and the like. Specifically, the simulation unit executes a conditional generative model. The conditional generative model is constructed by a deep neural network. The simulation unit receives a current state vector of a patient as an input. The state vector is a multidimensional numerical array including flags of age and medical history. Furthermore, the simulation unit receives an environmental condition vector as an input. The environmental condition vector is an array in which a distance of the family and a disaster occurrence situation are quantified. For example, the input is a numerical array in which a home end-of-life care wish flag is 1 and a family far away flag is 1. The conditional generative model maps these input vectors to a high-dimensional latent space. The conditional generative model outputs a time-series state transition probability matrix. Furthermore, the conditional generative model outputs a score array of recommended actions at each time step. The simulation unit transmits the score array to a natural language generation module. The natural language generation module converts the score array into a human-understandable text character string. The simulation unit extracts a scenario in which the score array exceeds a predetermined threshold. The simulation unit transmits the extracted scenario to the interface unit. The interface unit renders the scenario on a screen. Thereby, the system calculates a scenario branch in a high-dimensional condition space at high speed. The system presents comprehensive simulation results with low latency. As a result, the system realizes diverse risk predictions exceeding human cognitive limits. The present technology is applied to support for formulating an advance care plan in a nursing care facility or home medical care.

Step 7: Data linkage and security management. A data linkage unit performs API linkage with an external health management service, an electronic medical record system of a medical institution, a nursing care record system, a wearable terminal, and the like, automatically acquires data such as blood pressure, pulse, body temperature, number of steps, sleep time, fall history, hospital visit record, medication alert, rehabilitation progress, and nursing care record, and provides these data to the personal information management unit and the situation analysis unit. A security management unit is responsible for security and privacy protection of the entire system, includes functions such as encrypted communication, multi-factor authentication, access authority management, and audit log management, sets an appropriate information disclosure range for each related party such as the user himself/herself, the family, and a medical worker, and realizes strict information management. Specifically, the data linkage unit connects to an external system using an asynchronous communication protocol. The data linkage unit acquires time-series data from the external system. The time-series data includes raw data such as blood pressure and the number of steps. The data linkage unit executes an interpolation algorithm for missing values on the raw data. The interpolation algorithm includes linear interpolation and spline interpolation. The data linkage unit executes a filtering process for removing outliers. The data linkage unit converts the preprocessed data into a fixed-length tensor. The tensor is a two-dimensional numerical array consisting of the number of time steps and the number of features. The data linkage unit transmits the tensor to the situation analysis unit. On the other hand, the security management unit executes an advanced encryption algorithm. The encryption algorithm is, for example, a common key cryptosystem having a key length of 256 bits. The security management unit executes a token verification process in multi-factor authentication. The security management unit generates a bitmask indicating an access authority of the user. The security management unit dynamically controls access to data using the bitmask. Thereby, the system converts data of heterogeneous formats into a unified tensor representation. The system improves inference accuracy of an artificial intelligence model in a subsequent stage. Furthermore, the system prevents data falsification by encryption and access control. As a result, the system mathematically guarantees integrity and confidentiality of the entire system. The present technology is applied to a cloud platform handling highly confidential medical data.

### Specific Use Case

For example, assume a case where, in a household where only an elderly couple lives, a husband suddenly loses consciousness at home, and a spouse makes an emergency call using a terminal. At this time, hypertension, heart disease, and mild dementia are registered as past illnesses of the husband in the personal information management unit, and blood pressure and pulse, medication history, exercise habits, dietary habits, smoking history, drinking history, family structure, legal representative information, and "do not wish for life-prolonging treatment", "cardiopulmonary resuscitation is unnecessary", "refuse attachment of an artificial respirator", "give top priority to pain relief", "suspend determination of a treatment policy until the family arrives", and the like as the advance directive are registered in detail as daily health conditions. Furthermore, data such as a recent increase in blood pressure, difficulty in walking, nocturia, and an increase in fall risk are automatically acquired from an external health management service, an electronic medical record system of a medical institution, and a wearable terminal. Specifically, the system receives an emergency call trigger from the terminal. The emergency call trigger activates an event-driven architecture. The personal information management unit executes a distributed database system. The personal information management unit queries profile data of a corresponding user. The profile data includes a past illness flag and a time-series array of vital data. The personal information management unit combines the acquired data as a feature vector. The feature vector is composed of hundreds of dimensions of numerical elements. The personal information management unit caches the feature vector on a memory. The personal information management unit transfers the feature vector to the situation analysis unit at high speed. Thereby, the system executes parallel reading from the distributed database. The system shortens data acquisition latency in an emergency to a millisecond unit. As a result, the system enables real-time situation grasping. The present technology is applied to an initial response support system in emergency medical care.

The situation analysis unit receives an emergency call "the person himself/herself has collapsed and is unconscious" from the terminal, and comprehensively analyzes the data of the personal information management unit and the latest health monitoring data by the generative AI. The generative AI refers to the past illnesses and the advance directive of the person himself/herself, contact information of the family, legal representative information, the past communication history, medical guidelines, the social ethical codes, and the like, automatically determines a degree of urgency, and estimates a risk of myocardial infarction or stroke. Furthermore, considering that the person himself/herself clearly states in the advance directive that he/she "does not wish for cardiopulmonary resuscitation", that the family lives far away, that the spouse is upset, and the like, the generative AI immediately notifies an ambulance crew and a medical institution to the effect that "resuscitation measures are not performed and palliative care is prioritized". Specifically, the situation analysis unit executes a multimodal neural network. The situation analysis unit receives an embedding vector of a text input as an input. The embedding vector is, for example, a 512-dimensional numerical array. The situation analysis unit receives a time-series tensor of vital data as an input. The time-series tensor is an array indicating fluctuations in heart rate and blood pressure over the past 24 hours. The situation analysis unit receives a profile feature vector as an input. The multimodal neural network integrally calculates these heterogeneous data in a high-dimensional space. The multimodal neural network outputs a degree of urgency score. The degree of urgency score is a floating-point number from 0 to 1. Furthermore, the multimodal neural network outputs a probability distribution of a disease class. For example, the output is a numerical array indicating that a probability of myocardial infarction is 0.85 and a probability of stroke is 0.12. The situation analysis unit determines whether or not the degree of urgency score exceeds a predetermined threshold. The threshold is set to, for example, 0.8. When the degree of urgency score exceeds the threshold, the situation analysis unit activates an immediate notification module. The immediate notification module transmits an alert signal to an external emergency system. Thereby, the system integrally analyzes a plurality of types of data. The system classifies the degree of urgency with higher accuracy than rule-based determination based on a single data source. As a result, the system reduces false alarms and speeds up appropriate medical intervention. The present technology is applied to a triage automation system at the time of emergency transport.

The decision-making support unit executes an ethical decision-making algorithm by the generative AI, comprehensively considers the values and wishes of the person himself/herself, the intentions of the family, the situation of the medical site, the social ethical codes, and the like, and generates an optimal option. For example, the decision-making support unit presents options such as "do not perform cardiopulmonary resuscitation and give top priority to pain relief", "suspend determination of a treatment policy until the family arrives", "attachment of an artificial respirator is unnecessary", and "maximally respect the intentions of the person himself/herself and do not perform life-prolonging treatment", and explains the basis and reason for these options with high transparency in a natural language. For example, the decision-making support unit automatically generates an explanation such as "it is optimal to respect a natural course without performing cardiopulmonary resuscitation because it is described in the advance directive of the person himself/herself that he/she does not wish for life-prolonging treatment, and similar intentions have been confirmed from a past dialogue history with the family", or "it is recommended to temporarily suspend determination of a treatment policy and contact the family because the family is far away". Specifically, the decision-making support unit executes a reinforcement learning-based recommendation model. The decision-making support unit receives a disease probability distribution from the situation analysis unit as an input. The decision-making support unit receives a natural language embedding vector of the advance directive as an input. The decision-making support unit receives a constraint matrix of ethical codes as an input. The recommendation model generates a list of a plurality of action options based on these inputs. The recommendation model calculates a utility score for each option. The utility score is a numerical value calculated based on a reward function. Furthermore, the recommendation model outputs a generated text based on an attention weight map. The generated text is a character string explaining a reason for selection. The decision-making support unit extracts the top three options having the highest utility scores. The decision-making support unit transmits the extracted options to the interface unit. Thereby, the system derives an optimal solution satisfying complex constraint conditions at high speed. The system realizes comprehensive option evaluation exceeding human cognitive limits by inference of a neural network. As a result, the system significantly reduces a decision-making burden on a medical worker. The present technology is applied to automatic generation of a clinical pathway in end-of-life medical care.

The simulation unit sets diverse scenarios such as "a case where the person himself/herself falls at home at night and is semi-conscious", "a case where the family is staying overseas and cannot be contacted immediately", "a case where a hospitalization destination is restricted during an epidemic of a new infectious disease", "a case where it is necessary to receive medical care at an evacuation shelter when a disaster occurs",

"a case where a mass infection occurs in a nursing care facility", and "a case where the person himself/herself wishes to spend his/her last moments at home", presents a result of decision-making based on the values and wishes of the person himself/herself and an influence thereof in a natural language for each scenario, and enables the user and the family to prepare and review decision-making while experiencing a plurality of situations in advance through these simulations. Specifically, the simulation unit executes a Markov decision process model. The simulation unit searches a state space using a Monte Carlo tree search algorithm. The simulation unit receives an initial state vector as an input. The initial state vector is a numerical array indicating a current health condition of a patient. The simulation unit receives an environmental fluctuation parameter as an input. The environmental fluctuation parameter is a numerical value indicating an arrival time of the family or a degree of tightness of infectious disease beds. The Markov decision process model outputs a predicted probability of a final state in each scenario. The Markov decision process model outputs a Q-value matrix indicating an action value. The simulation unit generates a scenario tree based on the predicted probability. The simulation unit generates coordinate data of nodes and edges for rendering on a graphical user interface of a user terminal. The simulation unit transmits the coordinate data to the interface unit. Thereby, the system efficiently searches a vast state space. The system generates prediction results of diverse scenarios in real time while suppressing consumption of calculation resources. As a result, the system quantitatively visualizes a future risk. The present technology is applied to optimal allocation of medical resources and support for planning advance measures.

The interface unit is implemented on the terminal side, provides a natural language dialogue interface for performing two-way communication with the user himself/herself and the family, and when the user or the family inputs a question such as "what happens if I have a stroke?", "how to respond if the family is far away?", "what will be the treatment policy if I develop dementia?", "what kind of preparation is necessary if I want to spend my last moments at home?", or "what kind of procedure is necessary if I wish to enter a nursing care facility?", the generative AI explains medical and nursing care options and contents of legal procedures in an easy-to-understand manner in response to this, enables the user and the family to input their own intentions and wishes in a dialogue format, the input contents are immediately reflected in the personal information management unit, and the advance directive and a values profile are dynamically updated. Specifically, the interface unit executes a pipeline integrating a speech recognition model and a natural language understanding model. The interface unit receives speech waveform data from a user as an input. The speech waveform data is, for example, a digital signal with a sampling rate of 16 kilohertz. The interface unit converts the speech waveform data into a text character string. The natural language understanding model receives the text character string as an input. The natural language understanding model outputs an intent classification label. The intent classification label is an identifier indicating a category such as a question or an information update. Furthermore, the natural language understanding model outputs an extracted entity. The entity is character string data indicating a target disease or a desired care form. The interface unit automatically generates an update query for a database using the entity. The interface unit transmits the update query to the personal information management unit. Thereby, the system converts a natural language input, which is unstructured data, into structured data in real time. The system automates dynamic updating of the database. As a result, the system maintains data consistency and eliminates input errors. The present technology is applied to a hands-free information input system for the elderly.

The data linkage unit performs API linkage with an external health management service, an electronic medical record system of a medical institution, a nursing care record system, a wearable terminal, and the like, automatically acquires data such as blood pressure, pulse, body temperature, step count, sleep time, fall history, hospital visit record, medication alert, rehabilitation progress, and nursing care record, and provides these data to the personal information management unit and the situation analysis unit. Specifically, the data linkage unit cooperates with edge computing technology. The data linkage unit executes a data stream processing engine. The data linkage unit receives raw data from an external application programming interface. The raw data is described in a hierarchical text data format. The data linkage unit executes an interpolation algorithm for missing values on the raw data. The data linkage unit executes an outlier removal process based on a Z-score. The data linkage unit converts the processed data into a normalized time-series tensor. The time-series tensor is a two-dimensional array having dimensions of the number of time steps and the number of features. The data linkage unit caches the time-series tensor in an in-memory database. The data linkage unit provides the time-series tensor in response to a read request from the situation analysis unit. Thereby, the system intensively performs preprocessing in the data linkage unit. The system significantly reduces a calculation load of an artificial intelligence model in a subsequent stage. As a result, the system improves processing throughput of the entire system. The present technology is applied to a large-scale data collection infrastructure from IoT devices.

The security management unit is responsible for security and privacy protection of the entire system, includes functions such as encrypted communication, multi-factor authentication, access authority management, and audit log management, sets an appropriate information disclosure range for each related party such as the user himself/herself, the family, and a medical worker, and realizes strict information management. Specifically, the security management unit executes a public key cryptography method. The security management unit executes a role-based dynamic access control algorithm. The security management unit receives an authentication request of a user as an input. The authentication request includes a user identifier and a hash value of a password. Furthermore, the authentication request includes a feature vector of biometric authentication. The security management unit generates an access token based on these inputs. The access token is an encrypted character string with an expiration date. The security management unit outputs a bitmask indicating an authority level. The security management unit verifies the access token and the bitmask at the time of a data access request to each functional unit. The security management unit filters and returns only permitted data fields. Thereby, the system strictly controls data access at a hardware level and a software level. The system prevents unauthorized access. As a result, the system mathematically reduces a leakage risk of highly confidential medical data. The present technology is applied to a zero-trust security infrastructure of a medical information system.

Specific examples of a prompt sentence to be read by the generative AI include "82-year-old male, past illnesses are hypertension and heart disease, the person himself/herself stated in an advance directive that he does not wish for life-prolonging treatment, currently unconscious, what kind of response is optimal at the time of emergency transport, explain options and reasons thereof in a natural language in consideration of values of the person himself/herself and intentions of a family", "the person himself/herself has developed dementia and it is difficult to express intentions, simulate a flow of surrogate decision-making based on a past speech history, the intentions of the family, and instructions of a legal representative, and explain an optimal response policy and a basis thereof", "the person himself/herself wishes to spend his/her last moments at home, what kind of medical and nursing care system is necessary when the family is far away, specifically explain necessary procedures and preparations", and "when the person himself/herself refuses a blood transfusion for religious reasons, what kind of alternative treatment can be considered in an emergency, generate an explanatory text for a medical site and the family". Specifically, the system includes a prompt generation unit. The prompt generation unit executes a process of embedding structured data extracted from a database into a natural language prompt template. The prompt generation unit receives a patient profile vector as an input. The prompt generation unit receives a current context vector as an input. The prompt generation unit analyzes these vectors and outputs an optimized prompt character string. The prompt character string is expressed as an array of tokens. The prompt generation unit supplies the generated prompt character string to an input layer of a large language model. The large language model starts an inference process using a self-attention mechanism. Thereby, the system dynamically constructs a prompt. The system enables an artificial intelligence model to perform inference accurately reflecting the latest patient state. As a result, the system significantly reduces an occurrence probability of factual errors. The present technology is applied to accuracy improvement of a natural language generation system specialized for a medical domain.

Application Example 1 The flow of the specific processing in Application Example 1 will be described. Each part of the system described below is realized by the data processing device 12 and the smart device 14. Additionally, the data processing device 12 is referred to as a "server," and the smart device 14 is referred to as a "terminal."

### Embodiment for carrying out the present invention

An embodiment of the present invention is an advanced decision-making support system for realizing high-quality care and decision-making with a sense of consent while maximally respecting dignity and self-determination rights of a care receiver himself/herself and reducing a burden on a family and a nursing care staff in a nursing care facility or a home nursing care site. The system is configured by linking a server and a terminal, and is composed of a plurality of functional units such as a care receiver information management unit, a situation analysis unit, a nursing care decision-making support unit, a simulation unit, an interface unit, a data linkage unit, and a security management unit. Specifically, the system is constructed on a cloud computing environment. The system adopts a microservices architecture. Each functional unit of the system is deployed as an independent container. The container isolates hardware resources using virtualization technology. Each functional unit communicates asynchronously via a message queue. The message queue executes data transfer based on a publish-subscribe model. Thereby, the system prevents the entire system from stopping even when a load is concentrated on a specific functional unit. The system scales out each functional unit independently. As a result, the system dramatically improves scalability and fault tolerance. The present technology is applied to construction of a high-availability system in a large-scale medical and nursing care network.

The care receiver information management unit is realized on the server, and manages a medical history (hypertension, diabetes, heart disease, history of stroke, osteoporosis, presence or absence of dementia, past hospitalization history, surgery history, allergy information, infectious disease history, etc.), a nursing care history (past fall history, aspiration history, presence or absence of nighttime toilet assistance, frequency of meal assistance, rehabilitation implementation record, bedsore occurrence history, excretion trouble, medication error, presence or absence of nursing care refusal, daytime activity amount, recreation participation status, etc.), lifestyle habits (exercise habits, dietary habits, presence or absence of smoking/drinking, sleep pattern, daytime activity amount, hobbies/preferences, outing frequency, social participation status, etc.), values (wishing to avoid hospitalization, not wishing for life-prolonging treatment, giving top priority to pain relief, specific religious considerations, emphasizing visits with family, wishing to spend last moments at home, wishing for active treatment, wishing for a natural course, designation of a surrogate decision-maker when it is difficult to express intentions of the person himself/herself, etc.), intentions of a family (family lives far away and frequent visits are difficult, family is designated as a surrogate decision-maker, family wants to actively participate in a nursing care policy, opinions are divided among family members, wanting to reduce a mental burden on the family, family wants to give top priority to quality of life of the person himself/herself, etc.), legal information (presence or absence of an adult guardian, voluntary guardianship contract, executor, medical consent form, contents of an advance directive, declaration of death with dignity, designation of a surrogate decision-maker, medical expense payment method, nursing care insurance utilization status, etc.), a care plan (bathing frequency, rehabilitation frequency, medication management, meal contents, excretion assistance, outing support, recreation participation, presence or absence of visiting nursing, presence or absence of a visiting doctor, short stay utilization status, day service utilization status, nighttime monitoring system, etc.), vital data (blood pressure, pulse, body temperature, blood glucose level, oxygen saturation, body weight, respiratory rate, electrocardiogram data, sleep time, activity amount, etc.), behavior data from a wearable terminal (step count, fall detection, sleep time, activity amount, heart rate variability, position information, pressing history of an emergency call button, etc.), and input information by a nursing care staff or a family (nursing care record, diary, messages from the family, recent state and mood changes of the person himself/herself, new wishes and anxieties of the person himself/herself, findings of the nursing care staff, comments from a medical worker, etc.) of the care receiver himself/herself in an integrated manner. For example, when the nursing care staff inputs a daily nursing care record from the terminal, when the family updates the values and wishes of the person himself/herself from a smartphone, when the wearable terminal detects a nighttime fall, when the latest medical examination information is automatically acquired from a medical institution by API linkage, when the person himself/herself expresses his/her intentions by voice input saying "recently I have come to desire a natural course rather than active treatment", or when the family inputs "we wish for end-of-life care at home from now on", all information is accumulated in the server in real time, and is dynamically updated according to a change in a situation and a new input. Specifically, the care receiver information management unit executes a distributed graph database. The care receiver information management unit receives unstructured data and semi-structured data transmitted from various sensors and terminals as an input. The care receiver information management unit executes an entity extraction process using a natural language processing model. The care receiver information management unit executes relationship mapping between extracted entities. The care receiver information management unit outputs a knowledge graph data structure composed of nodes and edges. The node represents an entity such as a patient, a disease, or a wish. The edge represents a relationship such as suffering from or wishing for. The care receiver information management unit expands the knowledge graph on a memory. The care receiver information management unit responds to a complex query from the situation analysis unit in milliseconds. Thereby, the system integrates a wide variety of data as a knowledge graph. The system enables high-speed search for hidden correlations between data. As a result, the system provides a robust data infrastructure that supports inference accuracy of an artificial intelligence model. The present technology is applied to a next-generation electronic medical record system that integrally manages complex medical information.

The situation analysis unit is realized on the server, and analyzes a situation of the care receiver in an emergency or a normal time in real time. For example, when information such as "the person himself/herself fell in the toilet", "fever continues", "choked during a meal", "wandered at night", "a vital sensor detected an abnormal value", "the family contacted that there was a change in the state of the person himself/herself", "the nursing care staff reported a decrease in the consciousness level of the person himself/herself", "a medication error occurred", "excretion troubles occur frequently", or "a bedsore newly occurred" is input from the terminal, the situation analysis unit comprehensively analyzes the medical history, the nursing care history, the lifestyle habits data, the latest health monitoring data, the communication history from the family and the nursing care staff, the information from the medical site, and the like accumulated in the care receiver information management unit using the generative AI. For example, when a fall detection sensor is activated, the past fall history, the presence or absence of osteoporosis, the stability of vitals, the values of the person himself/herself "wishing to avoid hospitalization", the contact information of the family, and the current care plan are immediately referred to, the degree of urgency is automatically determined, and a risk of a bone fracture or a head trauma is estimated. When a fever is detected, past illnesses, an infectious disease risk, a medication status, a dehydration risk, the intentions of the family, a cooperation status with a medical institution, and the like are comprehensively determined, and a necessary response is derived. When there is nighttime wandering, the progression degree of dementia, a past wandering history, the wishes of the family, safety measures of the facility, position information by GPS, and the like are analyzed, and an increase in a wandering risk and the necessity of additional measures are determined. When aspiration during a meal occurs, a past aspiration history, a swallowing function evaluation, a meal form, findings of a nurse or a speech-language-hearing therapist, the wishes of the family, a cooperation status with a medical institution, and the like are comprehensively analyzed, and the necessity of first aid or a visit to a medical institution is determined. Specifically, the situation analysis unit executes an ensemble model combining a recurrent neural network for time-series data processing and a transformer model for text processing. The situation analysis unit receives a multidimensional time-series tensor from a sensor as an input. The multidimensional time-series tensor is a three-dimensional array including acceleration, angular velocity, and heart rate. The situation analysis unit receives a text embedding vector of a nursing care record as an input. The ensemble model integrates these inputs and outputs an anomaly detection score. Furthermore, the ensemble model outputs a state classification label. The state classification label indicates a category such as a fall, a fever, or wandering. The ensemble model outputs a certainty factor for the state classification label. The certainty factor is a numerical value from zero to one. The situation analysis unit determines whether or not the certainty factor exceeds a predetermined threshold value. When the certainty factor exceeds the threshold value, the situation analysis unit generates a corresponding alert event. The situation analysis unit transmits the alert event to the nursing care decision-making support unit. Thereby, the system integrates and analyzes multimodal data. The system identifies a complex abnormal state that is difficult to detect with single sensor data with high accuracy and low latency. As a result, the system prevents oversights at the nursing care site and improves safety. The present technology is applied to an automatic monitoring system in an elderly facility.

The nursing care decision-making support unit executes an ethical decision-making algorithm by the generative AI based on an analysis result obtained from the situation analysis unit and data of the care receiver information management unit, comprehensively considers values and wishes of the person himself/herself, intentions of a family, a care plan, a situation of a medical site, social ethical codes, and the like, and generates an optimal nursing care policy and care procedure. For example, individual and specific options are presented, such as "immediately contacting the family at the time of a fall and prioritizing follow-up observation in the facility", "contacting a medical institution at the time of a fever and thoroughly ensuring hydration and rest", "performing first aid at the time of aspiration during a meal and visiting a medical institution as necessary", "ensuring safety and reporting a situation to the family at the time of nighttime wandering", "strengthening home care when the person himself/herself does not wish for hospitalization", "adjusting medication to give top priority to pain relief", "avoiding specific treatment for religious reasons", "prompt medical cooperation and care plan review at the time of bedsore occurrence", "immediate reporting to a doctor and proposal of recurrence prevention measures at the time of a medication error occurrence", and "strengthening excretion care and explaining to the family at the time of frequent excretion troubles". The generative AI explains a basis and a reason for these options with high transparency in a natural language. For example, an explanation according to a situation is automatically generated, such as "since an intention to avoid hospitalization is registered as a wish of the person himself/herself, follow-up observation at home or in the facility in cooperation with the family is recommended", "when a risk of a bone fracture or a head trauma due to a fall is high, a prompt visit to a medical institution is recommended", "since a fever is recognized, contacting a medical institution and hydration are recommended in consideration of a risk of dehydration or an infectious disease", "since nighttime wandering occurs frequently, carrying a GPS terminal and strengthening a nighttime monitoring system are proposed", and "since a bedsore has newly occurred, contacting a medical institution and reviewing the care plan are recommended". Specifically, the nursing care decision-making support unit executes a rule-based inference engine and a deep learning-based generative model in a hybrid manner. The nursing care decision-making support unit receives an abnormal state label from the situation analysis unit as an input. The nursing care decision-making support unit receives a constraint condition vector of a patient extracted from a knowledge graph as an input. The constraint condition vector is a numerical array including a hospitalization refusal flag and a religious constraint flag. The hybrid model outputs a step array of a recommended care procedure. The hybrid model outputs a priority score for each step. Furthermore, the hybrid model outputs a text token sequence of an explanatory text. The nursing care decision-making support unit sorts the care procedure based on the priority score. The nursing care decision-making support unit push-notifies the sorted care procedure to a terminal of a nursing care staff via the interface unit. Thereby, the system achieves both safety assurance by strict rule-based processing and presentation of flexible countermeasures by the generative model. The system dramatically improves the quality and speed of decision-making at a medical and nursing care site. As a result, the system enables even an inexperienced staff to perform an appropriate response equivalent to that of an expert. The present technology is applied to a business support and education system in a nursing care facility.

The simulation unit virtually simulates various emergencies and nursing care and medical scenarios by the generative AI, sets various scenarios such as "when falling in the toilet at night", "when a high fever occurs", "when aspirating during a meal", "when wandering at night", "when the family is far away and cannot be contacted", "when a hospitalization destination is restricted during an epidemic of a new infectious disease", "when it is necessary to receive nursing care at an evacuation shelter when a disaster occurs", "when a mass infection occurs in a nursing care facility", "when the person himself/herself wishes to spend his/her last moments at home", "when a bedsore worsens", and "when medication errors occur successively", presents a result of decision-making based on values and wishes of the person himself/herself and a care plan and an influence thereof in a natural language for each scenario, enables a user, the family, and a nursing care staff to prepare and review decision-making while experiencing a plurality of situations in advance through these simulations, and automatically updates an advance directive, personal information, and the care plan based on a simulation result. Specifically, the simulation unit executes a virtual environment simulator applying digital twin technology. The simulation unit receives a current state vector of a patient as an input. The simulation unit receives a fluctuation parameter matrix of an environment as an input. The virtual environment simulator outputs a future state transition probability in each scenario. The virtual environment simulator outputs a risk score array. Furthermore, the virtual environment simulator outputs a natural language text summarizing the simulation result. The simulation unit extracts a scenario in which the risk score exceeds a specific threshold value. The simulation unit generates proposal data of a preventive measure for the extracted scenario. The simulation unit automatically updates care plan data of the care receiver information management unit using the proposal data. Thereby, the system calculates a future prediction in a high-dimensional parameter space at high speed. The system visualizes a complex risk that is difficult for a human to predict in advance. As a result, the system enables formulation of a proactive care plan. The present technology is applied to optimization of care management in a nursing care insurance system.

The interface unit is implemented on the terminal side, provides a natural language dialogue interface for performing two-way communication with the user himself/herself, the family, and the nursing care staff, and when a question such as "is hospitalization necessary for this fall?", "what are future recurrence prevention measures?", "what will be the treatment policy if the person himself/herself develops dementia?", "what kind of preparation is necessary if he/she wants to spend his/her last moments at home?", "what kind of procedure is necessary if he/she wishes to enter a nursing care facility?", "what are measures when nighttime wandering occurs frequently?", "how to respond when a bedsore worsens?", or "what are recurrence prevention measures when medication errors occur successively?" is input, the generative AI explains medical and nursing care options and contents of legal procedures in an easy-to-understand manner in response to this, enables the user, the family, and the nursing care staff to input their own intentions and wishes in a dialogue format, the input contents are immediately reflected in the care receiver information management unit, and the advance directive and the care plan are dynamically updated. Specifically, the interface unit executes a lightweight speech recognition model and an intent extraction model on a client side. The interface unit receives a voice input or a text input of a user. The intent extraction model analyzes the input and outputs a structured query object. The query object includes a key-value pair indicating a target disease or a requested action. The interface unit transmits the query object to a generative artificial intelligence on a server side. The interface unit renders a response text received from the server on a screen. Furthermore, the interface unit transmits the extracted intentions of the user to the care receiver information management unit. The care receiver information management unit updates a database using the received data. Thereby, the system provides an intuitive natural language interface. The system enables even a user with low information technology literacy to easily operate a complex system. As a result, the system lowers a hurdle of data input and maintains freshness and accuracy of information. The present technology is applied to a mobile application for the elderly and nursing care workers.

A data linkage unit links via API with an external health management service, an electronic medical record system of a medical institution, a nursing care record system, a wearable terminal, and the like, automatically acquires data such as blood pressure, pulse, body temperature, number of steps, sleep time, fall history, hospital visit record, medication alert, rehabilitation progress, nursing care record, electrocardiogram data, activity amount, GPS location information, and emergency call history, and provides these data to a care receiver information management unit and a situation analysis unit. Specifically, the data linkage unit executes a data pipeline orchestrator. The data linkage unit processes data collection from a plurality of external application programming interfaces asynchronously and in parallel. The data linkage unit receives raw data in various formats acquired from an external system as an input. The data linkage unit executes conversion processing of a data structure using a schema mapping algorithm. The data linkage unit executes synchronization processing of a time stamp. The data linkage unit executes noise removal filtering. The data linkage unit outputs a multidimensional time-series tensor having a unified schema. The data linkage unit streams the converted tensor to the care receiver information management unit and the situation analysis unit via a message queue. Thereby, the system automates data integration between heterogeneous systems. The system constructs a real-time data pipeline. As a result, the system guarantees that an artificial intelligence model always performs inference based on latest and clean data. The present technology is applied to a data infrastructure of a community-based comprehensive care system connecting medical institutions and nursing care facilities.

A security management unit is responsible for security and privacy protection of the entire system, includes functions such as encrypted communication, multi-factor authentication, access authority management, audit log management, data backup, information leakage detection, recording of usage history, terminal authentication, and access control for each user, sets an appropriate information disclosure range for each related person such as the user himself/herself, the family, the nursing care staff, and a medical worker, makes a medical history and values of the person himself/herself viewable only by the person himself/herself and the family, discloses information to a legal representative and a medical worker only within a necessary range, and realizes strict information management. Specifically, the security management unit executes a dynamic access control algorithm based on a zero trust architecture. The security management unit receives an internet protocol address of an access request source as an input. The security management unit receives a device fingerprint as an input. The security management unit receives biometric authentication data of a user as an input. The dynamic access control algorithm dynamically calculates and outputs a trust score based on these inputs. The dynamic access control algorithm outputs a determination result of access permission or denial. The security management unit determines whether or not the trust score falls below a predetermined threshold. When the trust score falls below the threshold, the security management unit transmits a prompt requesting additional multi-factor authentication to an interface unit. Furthermore, the security management unit records all access histories in a blockchain-based tamper-proof log system. Thereby, the system executes dynamic access control according to a context. The system provides tamper-proof log recording. As a result, the system maximizes resistance of the system against internal fraud and a cyberattack from the outside. The present technology is applied to a highly secure medical data sharing platform compliant with the Personal Information Protection Law.

As described above, in the embodiment of the present invention, with the generative AI as an essential element, comprehensive and flexible decision-making support such as integrated management of care receiver information, real-time analysis of a situation, ethical decision-making support, explanation and dialogue in a natural language, scenario simulation, dynamic update of an advance directive and a care plan, external data linkage, and security management is realized, and it is possible to reduce a burden on the family and the nursing care staff and provide high-quality care and decision-making with a sense of consent while maximizing respect for dignity and right to self-determination of the person himself/herself at a nursing care site. For example, it is possible to respond to various usage scenes such as: the nursing care staff can immediately confirm intentions of the person himself/herself using a terminal at the site when the person himself/herself suddenly changes; the family can participate in decision-making via a terminal even from a distance; a medical worker can determine a treatment policy while referring to the values and past declaration of intentions of the person himself/herself; a nursing care facility staff can share a change in a state of the person himself/herself with the family by inputting a daily care record into the system; AI automatically issues a risk alert and proposes an additional measure when a risk of night wandering or falling increases; and AI automatically proposes a recurrence prevention measure and automatically corrects the care plan when a problem such as a bedsore or a medication error occurs. Furthermore, the generative AI can continuously learn input contents of the user and a situation change, and evolve individually optimized decision-making support. Specifically, the entire system forms a feedback loop implementing a continuous learning framework. The system receives feedback data from the user as an input. The feedback data includes a history vector of approval or correction for the proposed care plan. The system executes fine-tuning processing of a weight parameter of a neural network using a backpropagation method. The system outputs an updated weight parameter matrix. The system seamlessly deploys the updated model to a production environment. The system improves next inference accuracy. Thereby, the system autonomously evolves the artificial intelligence model by utilizing data accumulated through operation. The system continuously improves personalization accuracy specialized for each user. As a result, the system increases value of the entire system over time. The present technology is applied to construction of a self-adaptive medical and nursing care artificial intelligence agent.

### System Configuration

The system according to the present embodiment includes a care receiver information management unit, a situation analysis unit, a nursing care decision-making support unit, a simulation unit, an interface unit, a data linkage unit, and a security management unit. The care receiver information management unit records in detail hypertension, diabetes, heart disease, history of stroke, osteoporosis, presence or absence of dementia, past hospitalization history, surgery history, allergy information, infectious disease history, and the like as a medical history of a care receiver himself/herself, accumulates past fall history, aspiration history, presence or absence of night toilet assistance, frequency of meal assistance, rehabilitation implementation record, bedsore occurrence history, excretion trouble, medication error, presence or absence of nursing care refusal, daytime activity amount, recreation participation status, and the like as a nursing care history, manages exercise habits, dietary habits, presence or absence of smoking/drinking, sleep pattern, daytime activity amount, hobbies/preferences, outing frequency, social participation status, and the like as lifestyle habits, records wanting to avoid hospitalization, not wishing for life-prolonging treatment, giving top priority to pain relief, specific religious consideration, emphasizing meeting with family, wanting to spend last moments at home, wishing for active treatment, desiring a natural course, designation of a surrogate decision-maker when it is difficult to express intentions of the person himself/herself, and the like as values, manages family lives far away and frequent meeting is difficult, family is designated as a surrogate decision-maker, family wants to actively participate in a nursing care policy, opinions are divided among family members, wanting to reduce a mental burden on family, family wants to give top priority to quality of life of the person himself/herself, and the like as intentions of a family, records presence or absence of an adult guardian, voluntary guardianship contract, executor, medical consent form, contents of an advance directive, declaration of death with dignity, designation of a surrogate decision-maker, medical expense payment method, nursing care insurance usage status, and the like as legal information, manages bathing frequency, rehabilitation frequency, medication management, meal contents, excretion assistance, outing support, recreation participation, presence or absence of visiting nursing, presence or absence of a visiting doctor, short stay usage status, day service usage status, night monitoring system, and the like as a care plan, collects blood pressure, pulse, body temperature, blood glucose level, oxygen saturation, body weight, respiratory rate, electrocardiogram data, sleep time, activity amount, and the like as vital data, automatically acquires number of steps, fall detection, sleep time, activity amount, heart rate variability, location information, pressing history of an emergency call button, and the like as behavior data from a wearable terminal, and manages a nursing care record, a diary, a message from family, a recent state and mood change of the person himself/herself, new wishes and anxiety of the person himself/herself, findings of nursing care staff, comments from medical worker, and the like as input information by a nursing care staff or a family in an integrated manner. For example, when the nursing care staff inputs a daily nursing care record from a terminal, when the family updates the values and wishes of the person himself/herself from a smartphone, when the wearable terminal detects a night fall, when latest medical examination information is automatically acquired from a medical institution by API linkage, when the person himself/herself expresses intentions by voice input saying "recently I have come to desire a natural course rather than active treatment", or when the family inputs "we wish for end-of-life care at home from now on", all information is accumulated in the server in real time, and is dynamically updated according to a change in a situation and a new input. Specifically, the system operates on a distributed computing environment. The care receiver information management unit is configured as a distributed database system. The care receiver information management unit converts various input text data and time-series numerical values into an embedding tensor on a high-dimensional vector space using a natural language processing model. The care receiver information management unit stores the converted embedding tensor in a vector database. The situation analysis unit includes a multimodal neural network. The situation analysis unit combines time-series vital data and a text embedding tensor to form an input feature amount. The nursing care decision-making support unit includes a large language model and a reinforcement learning agent. The simulation unit executes a Monte Carlo tree search algorithm. The interface unit communicates with a terminal using an asynchronous communication protocol. The data linkage unit normalizes raw data acquired from an external system. The security management unit protects data using an advanced encryption algorithm. With these configurations, the system enables integrated semantic analysis of heterogeneous mixed data beyond mere digitization of information. The system detects a state change of the care receiver with speed and accuracy exceeding human cognitive limits by vector calculation in a high-dimensional space. The system brings about a technical effect of improving efficiency of data management and improving accuracy of abnormality detection at a nursing care site. The system functions as a real-time decision-making support infrastructure in specific application fields such as an elderly nursing care facility and a home medical care network.

The situation analysis unit comprehensively analyzes the medical history, the nursing care history, the lifestyle habits data, the latest health monitoring data, the communication history from the family and the nursing care staff, the information from the medical site, and the like accumulated in the care receiver information management unit using the generative AI, and when information such as "the person himself/herself has fallen in the toilet", "fever continues", "choked during a meal", "wandered at night", "vital sensor detected an abnormal value", "family contacted that there was a change in the state of the person himself/herself", "nursing care staff reported a decrease in consciousness level of the person himself/herself", "medication error occurred", "excretion trouble occurs frequently", or "bedsore newly occurred" is input from the terminal, or when a fall detection sensor is activated, immediately refers to the past fall history, the presence or absence of osteoporosis, the stability of vitals, the values of the person himself/herself of "wanting to avoid hospitalization", the contact information of family, and the current care plan, automatically determines a degree of urgency, estimates a risk of fracture or head trauma, when fever is detected, comprehensively determines the past illness, the infectious disease risk, the medication status, the dehydration risk, the intentions of the family, the linkage status with a medical institution, and the like, derives a necessary response, when there is night wandering, analyzes the progression degree of dementia, the past wandering history, the wishes of the family, the safety measure of a facility, the location information by GPS, and the like, determines an increase in wandering risk and necessity of an additional measure, and when aspiration during a meal occurs, comprehensively analyzes the past aspiration history, the swallowing function evaluation, the meal form, the findings of a nurse or a speech-language-hearing therapist, the wishes of the family, the linkage status with a medical institution, and the like, and determines necessity of first aid or medical institution consultation. Specifically, the situation analysis unit executes a multimodal large language model based on a transformer architecture. The situation analysis unit receives text information, time-series vital data, and a sensor signal as input data. The situation analysis unit tokenizes the text information and converts it into a 512-dimensional word embedding vector. The situation analysis unit inputs the time-series vital data to a one-dimensional convolutional neural network and extracts a local feature map. The situation analysis unit converts the sensor signal into a spectrogram in a frequency domain. The situation analysis unit combines these heterogeneous data and generates an integrated feature tensor. For example, the input data to the situation analysis unit includes an embedding vector of text that the person himself/herself has fallen in the toilet, a time-series array of heart rate for the past 24 hours, and a waveform tensor from a 3-axis acceleration sensor. The situation analysis unit inputs the integrated feature tensor to a self-attention mechanism and calculates a hidden correlation between each data. The situation analysis unit outputs an emergency degree score, a risk probability distribution, and a category label of a recommended action via a softmax function of an output layer. For example, the situation analysis unit outputs an emergency degree score of 0.85, a fracture risk probability of 72 percent, and immediate contact to a medical institution which is a recommended action as an output. When the output emergency degree score exceeds a preset threshold, the situation analysis unit transmits an interrupt processing request to the subsequent nursing care decision-making support unit. Unlike conventional rule-based determination depending on human rules of thumb, the situation analysis unit evaluates a state using non-linear feature representation in a high-dimensional space. Thereby, the situation analysis unit instantly specifies a complex causal relationship between a minute vital change and a past medical history. The situation analysis unit exhibits a technical effect of shortening a delay of abnormality detection to a millisecond unit and significantly reducing a false detection rate. The situation analysis unit contributes to prevention of a fatal accident in specific application fields such as a remote monitoring system and a smart nursing care facility.

The nursing care decision-making support unit executes an ethical decision-making algorithm by the generative AI based on an analysis result obtained from the situation analysis unit and data of the care receiver information management unit, comprehensively considers values and wishes of the person himself/herself, intentions of a family, a care plan, a situation of a medical site, social ethical codes, and the like, generates an optimal nursing care policy and care procedure, presents individual and specific options such as immediately contacting family at the time of a fall and prioritizing follow-up observation in a facility, contacting a medical institution at the time of fever and thoroughly ensuring hydration and rest, performing first aid at the time of aspiration during a meal and consulting a medical institution as necessary, ensuring safety and reporting a situation to family at the time of night wandering, strengthening home care when the person himself/herself does not wish for hospitalization, drug adjustment for giving top priority to pain relief, avoidance of specific treatment due to religious reasons, prompt medical linkage and care plan review at the time of bedsore occurrence, immediate report to a doctor and proposal of a recurrence prevention measure at the time of medication error occurrence, and strengthening excretion care and explanation to family at the time of frequent excretion trouble, explains a basis and a reason for these options with high transparency in a natural language, and automatically generates an explanation according to a situation, such as recommends follow-up observation at home or in a facility in cooperation with family because intentions of wanting to avoid hospitalization are registered as wishes of the person himself/herself, recommends prompt medical institution consultation when a risk of fracture or head trauma due to a fall is high, recommends contact to a medical institution and hydration in consideration of a risk of dehydration or infectious disease because fever is recognized, proposes carrying a GPS terminal and strengthening a night monitoring system because night wandering occurs frequently, and recommends contact to a medical institution and review of a care plan because a bedsore newly occurred. Specifically, the nursing care decision-making support unit includes a decision-making neural network optimized by reinforcement learning. The nursing care decision-making support unit receives the emergency degree score and the risk probability distribution output from the situation analysis unit as an input. The nursing care decision-making support unit further combines a values vector and a care plan tensor acquired from the care receiver information management unit as an input feature amount. The nursing care decision-making support unit inputs these input data to a multilayer perceptron and calculates an expected reward value for a plurality of action candidates. The nursing care decision-making support unit uses a multi-objective optimization function quantifying a degree of conformity to wishes of the person himself/herself, medical safety, and a degree of burden reduction on family as a reward function. The nursing care decision-making support unit selects an action candidate that maximizes the calculated expected reward value. The nursing care decision-making support unit maps the selected action candidate to a prompt space of a large language model. The large language model executes autoregressive decoding processing and generates an explanation text in a natural language. For example, the input to the nursing care decision-making support unit includes an emergency degree score of 0.6, hospitalization avoidance priority which is a values vector, and home care wish which is a family intention vector. The nursing care decision-making support unit outputs follow-up observation at home which is an action label and a character string "we recommend follow-up observation at home in consideration of intentions of hospitalization avoidance of the person himself/herself and stability of vitals" which is a generated text as an output. The nursing care decision-making support unit transmits the output action label and generated text to the interface unit and triggers rendering processing on a screen. The nursing care decision-making support unit solves a subjective judgment process of a human by converting it into a mathematical optimization problem on a multidimensional vector space. Thereby, the nursing care decision-making support unit processes complexly intertwined ethical constraints and medical requirements without contradiction. The nursing care decision-making support unit brings about a technical effect of deriving an optimal solution at high speed while ensuring transparency of decision-making and suppressing consumption of computer resources. The nursing care decision-making support unit functions as a business support tool for a care manager in specific application fields such as a visiting nursing care station and a community comprehensive support center.

The simulation unit virtually simulates various emergencies and nursing care and medical scenarios by the generative AI, sets various scenarios such as when falling in a toilet at night, when high fever occurs, when aspirating during a meal, when there is night wandering, when family is far away and cannot be contacted, when a hospitalization destination is restricted during an epidemic of a new infectious disease, when it is necessary to receive nursing care at an evacuation shelter at the time of disaster occurrence, when mass infection occurs in a nursing care facility, when the person himself/herself wishes to spend last moments at home, when a bedsore worsens, and when medication errors occur successively, presents a result of decision-making based on the values and wishes of the person himself/herself and the care plan and an influence thereof in a natural language for each scenario, enables the user, the family, and the nursing care staff to prepare and review decision-making while experiencing a plurality of situations in advance through these simulations, and automatically updates the advance directive, the personal information, and the care plan based on a simulation result. Specifically, the simulation unit executes a probabilistic state transition model based on a Markov decision process. The simulation unit sets a current state vector of the care receiver as an initial value. The simulation unit probabilistically generates a plurality of events that can occur in the future using a random number generator. The simulation unit predicts a state change at the time of occurrence of each event in a time-series manner using a recurrent neural network. The simulation unit inputs the predicted future state tensor to the nursing care decision-making support unit and calculates a virtual countermeasure. The simulation unit evaluates thousands of patterns of scenario branches at high speed on a parallel computing node using a Monte Carlo tree search algorithm. For example, the input to the simulation unit includes a current health condition vector, an environment variable tensor, and a simulation period of 30 days. The simulation unit outputs an occurrence probability score of each scenario, a predicted value of a final health condition, and a recommended advance preparation list as an output. The simulation unit extracts a scenario in which the output predicted value exceeds a specific danger threshold and automatically updates a flag in a database of the care receiver information management unit. The simulation unit simulates dynamic state transition in a virtual space inside a computer, not mere statistical aggregation of past data. Thereby, the simulation unit verifies in advance a response of the system to a fatal situation that cannot be tested in the real world. The simulation unit exhibits a technical effect of improving prediction accuracy for an unknown risk and enhancing robustness of the entire system. The simulation unit is utilized as a data-driven prediction tool in specific application fields such as formulation of a business continuity plan of a nursing care facility and end-of-life planning of an individual.

The interface unit is implemented on the terminal side, provides a natural language dialogue interface for performing two-way communication with the user himself/herself, the family, and the nursing care staff, and when a question such as "is hospitalization necessary due to this fall?", "what is a future recurrence prevention measure?", "what will be a treatment policy if the person himself/herself develops dementia?", "what kind of preparation is necessary if wanting to spend last moments at home?", "what kind of procedure is necessary if wishing to enter a nursing care facility?", "what is a measure when night wandering occurs frequently?", "how to respond when a bedsore worsens?", or "what is a recurrence prevention measure when medication errors occur successively?" is input, the generative AI explains options regarding medical and nursing care and contents of legal procedures in an easy-to-understand manner in response to this, enables the user, the family, and the nursing care staff to input their own intentions and wishes in a dialogue format, and the input contents are immediately reflected in the care receiver information management unit, and the advance directive and the care plan are dynamically updated. Specifically, the interface unit includes a natural language processing engine and a voice recognition module. The interface unit converts an analog voice signal acquired from a microphone of the terminal into digital waveform data. The interface unit converts the digital waveform data into a text character string using a hidden Markov model and a deep learning-based acoustic model. The interface unit tokenizes the text character string and shapes it into a prompt format that can be input to a large language model. The interface unit transmits the shaped prompt to the nursing care decision-making support unit on the server side. The interface unit converts a response text received from the server into a voice signal using a voice synthesis engine and outputs it from a speaker. For example, the input to the interface unit includes voice waveform data of "what is a measure when night wandering occurs frequently?" and an identification ID of a user. The interface unit outputs structured data describing a measure plan and a rendering tree for screen display as an output. The interface unit extracts a text of new wishes and values input from the user, calls an update API of the care receiver information management unit, and rewrites a record of a database. Unlike a conventional static graphical user interface, the interface unit executes dynamic dialogue state management retaining a context on a memory. Thereby, the interface unit enables even an elderly person unfamiliar with computers and a busy nursing care staff to intuitively operate a complex system. The interface unit brings about a technical effect of reducing an input error rate and significantly shortening operation time required for data input. The interface unit realizes seamless linkage between human and machine in specific application fields such as a control interface of a smart speaker or a nursing care robot.

A data linkage unit links via API with an external health management service, an electronic medical record system of a medical institution, a nursing care record system, a wearable terminal, and the like, automatically acquires data such as blood pressure, pulse, body temperature, number of steps, sleep time, fall history, hospital visit record, medication alert, rehabilitation progress, nursing care record, electrocardiogram data, activity amount, GPS location information, and emergency call history, and provides these data to a care receiver information management unit and a situation analysis unit. Specifically, the data linkage unit functions as middleware managing a plurality of asynchronous communication threads. The data linkage unit periodically transmits a request to an endpoint of an external system. The data linkage unit converts raw data in various formats received from the external system into a standardized data schema inside the system. The data linkage unit executes data cleansing processing and performs interpolation of a missing value and filtering of an abnormal value. The data linkage unit applies synchronization processing of a time stamp and unification processing of a sampling rate to time-series data. The data linkage unit temporarily stores processed data in a buffer on a memory as a multidimensional tensor. For example, the input to the data linkage unit includes 100-hertz electrocardiogram waveform data transmitted from a wearable terminal and prescription data in a text format acquired from an electronic medical record system. The data linkage unit outputs a normalized heart rate variability feature amount vector and an embedding tensor of a drug code as an output. The data linkage unit asynchronously transfers the output tensor data to the situation analysis unit via a message queuing system. The data linkage unit absorbs inconsistency of data formats between heterogeneous systems by an automatic conversion algorithm inside a computer. Thereby, the data linkage unit completely eliminates work of manually transcribing data by a human. The data linkage unit exhibits a technical effect of minimizing latency of data integration processing and ensuring real-time property of the entire system. The data linkage unit functions as a data hub in specific application fields such as a medical information linkage network and a personal health record integration infrastructure.

A security management unit is responsible for security and privacy protection of the entire system, has functions such as encrypted communication, multi-factor authentication, access authority management, audit log management, data backup, information leakage detection, recording of a usage history, terminal authentication, and access control for each user, sets an appropriate information disclosure range for each related person such as a user himself/herself, a family, a nursing care staff, and a medical worker, makes a medical history and values of the person himself/herself viewable only by the person himself/herself and the family, and realizes strict information management such as disclosing information to a legal representative and the medical worker only within a necessary range. Specifically, the security management unit includes an authentication and authorization module based on a zero-trust architecture. The security management unit applies end-to-end encryption using a transport layer security protocol to all data communications in the system. The security management unit executes encryption during data storage using an advanced encryption standard algorithm for personal information and medical data stored in a database. The security management unit verifies a digital certificate based on a public key cryptography system when receiving an access request from a user. The security management unit further executes a multi-factor authentication process using biometric authentication data and a one-time password. The security management unit dynamically determines an access authority for a requested resource by referring to a role-based access control list. For example, an input to the security management unit includes an access request packet, a session token, and a data ID of a request target transmitted from a terminal of the nursing care staff. The security management unit outputs a Boolean value indicating access permission and a session key for decryption as an output. When access is denied, the security management unit transmits an alert signal to an intrusion detection system and temporarily blocks communication from the IP address. Unlike conventional boundary defense type security, the security management unit verifies all transactions in units of micro-segmentation. Thereby, the security management unit minimizes an information leakage risk due to an internal crime or horizontal deployment of malware. The security management unit brings about a technical effect of dramatically improving protection strength of confidential data and realizing safe data management satisfying compliance requirements. The security management unit provides a robust foundation in specific application fields such as a cloud system of a medical institution and an administrative platform handling highly confidential personal information.

Specific examples of a prompt sentence to be read by generative AI when implementing the present invention include "An 85-year-old woman, care level 2, history of hypertension and osteoporosis, a fall is detected during night toilet assistance. The person herself has intentions to avoid hospitalization. There is a history of two falls in the past, neither of which led to a serious matter. Current vitals are stable. A family lives far away. For this fall, explain an optimal response policy and a reason thereof in a natural language in consideration of values of the person herself and intentions of the family", "A care receiver has a fever, and has diabetes and heart disease as past illnesses. The person himself/herself does not wish for active treatment. A family wishes to visit a medical institution. Explain what kind of response is optimal, options, and a basis", "The person himself/herself wanders at night, and progression of dementia is suspected. Propose a future countermeasure plan in consideration of a past wandering history, wishes of a family, and safety measures of a facility", and "Aspiration occurs during a meal, and explain first aid and a future care plan review plan based on swallowing function evaluation and a past aspiration history". Specifically, the system includes a template engine for dynamically generating these prompt sentences. The template engine inserts structured data extracted from a care receiver information management unit into a placeholder of a natural language. The system converts the generated prompt sentence into a token sequence suitable for an input layer of a large language model. The system efficiently divides an unknown word and a technical term into subwords using a byte-pair encoding algorithm during tokenization. The system maps the token sequence to a 512-dimensional embedding vector space and adds positional encoding. For example, input data to the system is a data object including key-value pairs of age 85, gender female, care level 2, past illness hypertension, and event night fall. The system outputs a natural language prompt character string integrating these pieces of information and a numerical tensor obtained by converting the prompt character string as an output. The system inputs the output numerical tensor to an encoder block of a transformer model and causes a self-attention mechanism to execute context analysis. The system adopts a dynamic prompt construction algorithm considering context dependency instead of simple combination of fixed character strings. Thereby, the system enables the large language model to accurately capture nuances of a situation and suppress unfounded output. The system exhibits a technical effect of improving inference accuracy and enhancing reliability of a generated answer. The system realizes high-quality text generation in specific application fields such as a medical diagnosis support system and customer support AI involving complex conditional branching.

### Implementation Steps

Step 1: Collection and Registration of Care Receiver Information A nursing care staff, a family, and a medical worker use a terminal to accumulate a medical history (hypertension, diabetes, heart disease, history of stroke, osteoporosis, presence or absence of dementia, past hospitalization history, surgery history, allergy information, infectious disease history, etc.), a nursing care history (fall history, aspiration history, presence or absence of night toilet assistance, frequency of meal assistance, rehabilitation record, bedsore occurrence history, excretion trouble, medication error, presence or absence of nursing care refusal, daytime activity amount, recreation participation status, etc.), lifestyle habits (exercise habits, dietary habits, presence or absence of smoking/drinking, sleep pattern, hobbies/preferences, outing frequency, social participation status, etc.), values (wishing to avoid hospitalization, not wishing for life-prolonging treatment, giving top priority to pain relief, religious consideration, emphasizing meeting with family, wishing to spend last moments at home, wishing for active treatment, wishing for natural course, designation of surrogate decision maker, etc.), intentions of a family (living far away, designation of surrogate decision maker, involvement in nursing care policy, difference of opinion among family members, wishing to reduce mental burden, emphasizing quality of life, etc.), legal information (adult guardian, voluntary guardianship contract, executor, medical consent form, advance directive, declaration of death with dignity, designation of surrogate decision maker, medical expense payment method, nursing care insurance usage status, etc.), a care plan (bathing frequency, rehabilitation frequency, medication management, meal content, excretion assistance, outing support, recreation participation, visiting nursing, visiting doctor, short stay, day service, night monitoring system, etc.), vital data (blood pressure, pulse, body temperature, blood glucose level, oxygen saturation, body weight, respiratory rate, electrocardiogram data, sleep time, activity amount, etc.), behavior data from a wearable terminal (number of steps, fall detection, sleep time, activity amount, heart rate variability, position information, emergency call button pressing history, etc.), and input information by the nursing care staff or the family (nursing care record, diary, communication matter, state and mood change of the person himself/herself, new wishes and anxieties, staff findings, medical worker comments, etc.) of a care receiver himself/herself in a care receiver information management unit of a server. Specifically, in this step, the system executes a distributed data collection agent on each terminal. The data collection agent asynchronously acquires text data input via a user interface and sensor data received from the wearable terminal via wireless communication. The data collection agent temporarily buffers the acquired raw data on a local memory and reduces a payload size using a data compression algorithm. The data collection agent transmits the compressed data to the care receiver information management unit on the server side via a secure communication channel. The care receiver information management unit decompresses the received data and executes validation processing of a data schema. The care receiver information management unit applies natural language processing to the text data to extract keywords and link entities. The care receiver information management unit applies a noise removal filter to time-series vital data to calculate a feature vector. For example, an input to the system includes a text "there is a decrease in appetite today" input by the nursing care staff and a heart rate array at 1-minute intervals from the wearable terminal. The system outputs a statistical feature vector including an average value and variance of the heart rate and the decrease in appetite which is an extracted entity tag as an output. The system inserts the output structured data and feature vector into corresponding tables of a relational database and a vector database. The system constructs a data pipeline that seamlessly integrates manual data input by a human and automatic collection from an IoT device. Thereby, the system prevents data loss and always maintains the latest state. The system brings about a technical effect of enhancing comprehensiveness of data collection and improving quality of input data in subsequent analysis processing. The system functions as an integrated information management foundation in specific application fields such as a large-scale nursing care facility network and a wide-area cooperation system for home medical care.

Step 2: Detection of Situation Change and Data Linkage Abnormalities and daily changes such as a fall, a fever, a vital abnormality, wandering, aspiration, a medication error, an excretion trouble, and bedsore occurrence are automatically detected from a wearable terminal, a nursing care record system, an electronic medical record system, a health management service, and the like, and a nursing care staff or a family manually inputs a situation change from a terminal, whereby a data linkage unit provides these pieces of information to a care receiver information management unit and a situation analysis unit in real time. Specifically, in this step, the data linkage unit executes an event-driven stream processing engine. The stream processing engine stores time-series data continuously flowing from a plurality of data sources in an in-memory window buffer. The stream processing engine applies real-time aggregation and pattern matching processing to a data stream within a certain time frame using a sliding window method. The stream processing engine uses a machine learning model based on an isolation forest or an autoencoder as an anomaly detection algorithm. The stream processing engine calculates whether an input data point statistically significantly deviates from a normal distribution. For example, an input to the stream processing engine includes continuous tensor data of a 3-axis acceleration sensor transmitted every second from the wearable terminal. The stream processing engine outputs an anomaly score for each data point and an event trigger signal when an anomaly is detected as an output. When the anomaly score exceeds a preset threshold value, the stream processing engine immediately issues an interrupt request to the situation analysis unit and transfers a related data snapshot. The system executes continuous stream analysis on a memory instead of periodic data confirmation by batch processing. Thereby, the system detects a critical event such as a fall or a sudden vital change with a delay in units of milliseconds. The system exhibits a technical effect of maximizing real-time performance of anomaly detection and preventing aggravation due to a delay in response. The system functions as a monitoring system that ensures life safety in specific application fields such as patient monitoring in an intensive care unit and a watching service for elderly people living alone.

Step 3: Situation Analysis and Decision-Making Support Prompt Generation by Generative AI A situation analysis unit comprehensively refers to a medical history, a nursing care history, lifestyle habits, values, intentions of a family, legal information, a care plan, vital data, behavior data, a nursing care record, and the like accumulated in a care receiver information management unit, and generates a prompt for situation analysis and decision-making support to generative AI. Specific examples of a prompt sentence include "An 85-year-old woman, care level 2, history of hypertension and osteoporosis, a fall is detected during night toilet assistance. The person herself has intentions to avoid hospitalization. There is a history of two falls in the past, neither of which led to a serious matter. Current vitals are stable. A family lives far away. For this fall, explain an optimal response policy and a reason thereof in a natural language in consideration of values of the person herself and intentions of the family", and "A care receiver has a fever, and has diabetes and heart disease as past illnesses. The person himself/herself does not wish for active treatment. A family wishes to visit a medical institution. Explain what kind of response is optimal, options, and a basis". Specifically, in this step, the situation analysis unit executes a knowledge graph-based context extraction module. The context extraction module issues a query to a database of the care receiver information management unit triggered by a detected abnormal event. The context extraction module searches on a graph database and extracts nodes such as a medical history, values, and latest vital data related to the care receiver. The context extraction module integrates the extracted node group to construct a multidimensional context tensor. The situation analysis unit inputs the constructed context tensor to a transformer model for prompt generation. The transformer model weights information most relevant to a current abnormal event using an attention mechanism. For example, an input to the situation analysis unit includes a night fall which is an abnormal event and an attribute vector group such as osteoporosis and a wish to avoid hospitalization extracted from the graph database. The situation analysis unit outputs a natural language prompt character string logically configuring these pieces of information and a token embedding matrix which is an internal representation thereof as an output. The situation analysis unit passes the output prompt character string to an input interface of a large language model provided in a subsequent nursing care decision-making support unit. The system adopts a dynamic context construction algorithm using a knowledge graph instead of simple filling in a template. Thereby, the system filters only information truly necessary for current situation judgment from a huge amount of personal data and presents the information to the AI. The system brings about a technical effect of avoiding a limitation of a context window of the large language model and improving inference accuracy and processing efficiency. The system realizes advanced situation understanding in specific application fields such as emergency transport triage of a patient having a complex medical history and a personalized medical consultation system.

Step 4: Presentation and Explanation of Optimal Nursing Care Policy and Care Procedure by Generative AI A nursing care decision-making support unit executes an ethical decision-making algorithm by generative AI, comprehensively considers values and wishes of a person himself/herself, intentions of a family, a care plan, a situation of a medical site, social ethical codes, and the like, generates an optimal nursing care policy and care procedure, presents individual specific options such as immediately contacting the family and prioritizing follow-up observation in a facility at the time of a fall, contacting a medical institution and thoroughly ensuring hydration and rest at the time of a fever, performing first aid and visiting a medical institution as necessary at the time of aspiration during a meal, ensuring safety and reporting a situation to the family at the time of night wandering, strengthening home care when the person himself/herself does not wish for hospitalization, adjusting medication to give top priority to pain relief, avoiding specific treatment due to religious reasons, rapid medical cooperation and care plan review at the time of bedsore occurrence, immediate reporting to a doctor and proposal of a recurrence prevention measure at the time of medication error occurrence, and strengthening excretion care and explanation to the family at the time of frequent excretion trouble, and explains a basis and a reason for these options with high transparency in a natural language. Specific examples of a prompt sentence include "Since an intention to avoid hospitalization is registered as a wish of the person himself/herself, follow-up observation at home or in a facility in cooperation with a family is recommended", "When a risk of a fracture or a head injury due to a fall is high, prompt visit to a medical institution is recommended", and "Since a fever is recognized, contacting a medical institution and hydration are recommended in consideration of a risk of dehydration or an infectious disease". Specifically, in this step, the nursing care decision-making support unit executes a fine-tuned large language model specialized for medical and nursing care. The large language model is composed of a deep neural network having billions of parameters. The nursing care decision-making support unit tokenizes the prompt character string generated in Step 3 and supplies it to an input layer of the model. The large language model calculates a hidden state vector for an input context through an encoder and decoder architecture. The large language model autoregressively generates a word sequence with the highest probability using a beam search algorithm. The nursing care decision-making support unit applies a rule-based safety filter to the generated text to verify whether a medically inappropriate instruction is included. For example, an input to the nursing care decision-making support unit is a prompt tensor including conditions of fall detection, vital stability, and a wish to avoid hospitalization. The nursing care decision-making support unit outputs an action label recommending follow-up observation in a facility and a natural language text explaining a medical and ethical basis thereof as an output. The nursing care decision-making support unit packages the output action label and explanation text as structured data and transmits it to an interface unit. The system executes evaluation of a complex trade-off performed by a human expert as tacit knowledge on a computer as an optimization problem of a probability distribution in a high-dimensional space. Thereby, the system instantly derives objective decision-making that achieves both personal values and medical validity. The system exhibits a technical effect of eliminating variations in personal judgment and achieving individual optimization while standardizing quality of care. The system complements knowledge of an expert in specific application fields such as automation of night on-call response in a nursing care facility and an education support system for a new nursing care staff.

Step 5: Dialogue with User, Family, and Nursing Care Staff and Intention Reflection Via an interface unit, a user himself/herself, a family, and a nursing care staff can confirm options and explanations presented by generative AI on a terminal, and input an additional question and intention declaration in a natural language. For example, in response to a question such as "is hospitalization necessary for this fall?", "what is a future recurrence prevention measure?", "what will be a treatment policy when the person himself/herself develops dementia?", "what kind of preparation is necessary when wishing to spend last moments at home?", "what is a countermeasure when night wandering frequently occurs?", "how to respond when a bedsore worsens?", and "what is a recurrence prevention measure when medication errors occur successively?", the generative AI explains options for medical and nursing care and contents of legal procedures in an easy-to-understand manner, input contents are immediately reflected in a care receiver information management unit, and an advance directive and a care plan are dynamically updated. Specifically, in this step, the interface unit executes a stateful dialogue management module. The dialogue management module holds a series of exchanges with a user as a session on a memory and maintains continuity of context. The interface unit receives a natural language question text transmitted from a terminal of the user and inputs it to an intent classification neural network. The intent classification neural network specifies an intent of the question and outputs a corresponding intent vector. The interface unit combines the intent vector and a past dialogue history tensor and inputs them to a language model for response generation. For example, an input to the interface unit includes a text character string "is hospitalization necessary for this fall?" from the user and a current session state vector. The interface unit outputs a response text "hospitalization is unnecessary because vitals are stable" and an emotion score estimating an anxiety level of the user as an output. When the user inputs a new intention, the interface unit structures the content using an information extraction algorithm, automatically generates a database update query of the care receiver information management unit, and executes it. The system constructs a dynamic dialogue system that understands context and updates a state instead of a simple question-and-answer chatbot. Thereby, the system enables the user to interact with a complex system through a natural conversation. The system brings about a technical effect of lowering a psychological hurdle of the user and accurately reflecting potential wishes and values in the system. The system realizes smooth information sharing in specific application fields such as a communication support tool between a patient and a doctor and a situation reporting system to a family in a remote place.

Step 6: Scenario Simulation and Advance Preparation by Generative AI A simulation unit virtually simulates various scenarios by generative AI, such as when falling in a toilet at night, when a high fever occurs, when aspiration occurs during a meal, when wandering occurs at night, when a family is far away and cannot be contacted, when a hospitalization destination is restricted during an epidemic of a new infectious disease, when it is necessary to receive nursing care at an evacuation shelter when a disaster occurs, when a mass infection occurs in a nursing care facility, when a person himself/herself wishes to spend last moments at home, when a bedsore worsens, and when medication errors occur successively, presents a result of decision-making based on values and wishes of the person himself/herself and a care plan and an influence thereof in a natural language for each scenario, enables a user, the family, and a nursing care staff to prepare and review decision-making while experiencing a plurality of situations in advance through these simulations, and automatically updates an advance directive, personal information, and the care plan based on a simulation result. Specific examples of a prompt sentence include "When the person himself/herself falls in a toilet at night, explain an optimal response and a basis thereof in consideration of a past fall history, presence or absence of osteoporosis, intentions of a family, and a care plan", and "When a high fever occurs, explain a response plan and a reason thereof in consideration of a past illness, wishes of a family, and a cooperation status with a medical institution". Specifically, in this step, the simulation unit executes a parameterized scenario generation engine. The scenario generation engine reads a current health condition vector and an environment variable tensor of a care receiver as initial conditions. The simulation unit constructs a probability model of an event that can occur in the future based on a statistical distribution extracted from a past incident database. The simulation unit generates thousands of patterns of virtual time evolution scenarios on a parallel computing cluster using a Markov chain Monte Carlo method. The simulation unit calls an inference model of a nursing care decision-making support unit in each scenario and causes it to calculate an optimal countermeasure under a virtual situation. For example, an input to the simulation unit includes a current care level, a past illness vector, and a virtual event trigger of high fever occurrence. The simulation unit outputs a recommended countermeasure when a virtual event occurs, a predicted deterioration probability of a health condition, and a list of necessary advance preparation items as an output. The simulation unit aggregates output simulation results, generates a correction plan of a preventive care plan for a high-risk scenario, and feeds it back to a care receiver information management unit. The system executes dynamic future prediction combining probabilistic simulation and inference of a deep learning model instead of prediction based on a static rule. Thereby, the system comprehensively evaluates various risk scenarios exceeding human imagination. The system exhibits a technical effect of enabling conversion from reactive care to preventive care and mathematically reducing an occurrence rate of a serious accident. The system supports data-driven strategy planning in specific application fields such as benefit optimization simulation of nursing care insurance and a risk management system for facility operation.

Step 7: Security Management and Access Control A security management unit is responsible for security and privacy protection of the entire system, has functions such as encrypted communication, multi-factor authentication, access authority management, audit log management, data backup, information leakage detection, recording of a usage history, terminal authentication, and access control for each user, sets an appropriate information disclosure range for each related person such as a user himself/herself, a family, a nursing care staff, and a medical worker, makes a medical history and values of the person himself/herself viewable only by the person himself/herself and the family, and realizes strict information management such as disclosing information to a legal representative and the medical worker only within a necessary range. Specifically, in this step, the security management unit executes a dynamic policy evaluation engine. The policy evaluation engine functions as a proxy that intercepts all data access requests in the system. The security management unit generates a context vector including an IP address of an access request source, a fingerprint of a device, a role of a user, and a confidentiality level of requested data. The security management unit inputs the context vector to a machine learning-based anomaly detection model and scores validity of an access request in real time. The security management unit permits query execution to an encrypted database only when a score satisfies a threshold value. For example, an input to the security management unit includes an access request packet from an external network and an authentication token. The security management unit outputs an access permission flag and a transaction record for an audit log as an output. The security management unit asynchronously writes the output transaction record to a distributed ledger having a tamper-proof function. Unlike a conventional method depending on static password authentication, the system implements a zero-trust model that multidimensionally evaluates a context at the time of access. Thereby, the system blocks unauthorized access even by an attacker who has stolen authentication information of a legitimate user with high accuracy. The system brings about a technical effect of maximizing security strength in handling highly confidential data such as medical information and ensuring reliability of the system. The system provides a safe data distribution foundation in specific application fields such as a wide-area medical information network and a national-level health information management platform.

### Specific Use Case

For example, assume a case where a care receiver is admitted to an elderly care facility, has a nursing care level of 2, has hypertension and osteoporosis as past illnesses, has a history of two falls in the past, and wishes such as "want to avoid hospitalization as much as possible", "want to give top priority to pain relief", and "want to emphasize visiting with family" are registered in an advance directive as values of the person himself/herself. The family lives far away, and although daily visits are difficult, they have intentions to actively participate in important decision-making. At the facility, a walking state and vital data (blood pressure, pulse, body temperature, number of steps, sleep time, etc.) of the person himself/herself are constantly monitored using a wearable terminal, and a nursing care staff inputs daily nursing care records, a state of the person himself/herself, a situation of meal assistance and excretion assistance, progress of rehabilitation, presence or absence of night toilet assistance, and the like from a terminal. One night, a fall detection sensor was activated on the way to the toilet, and the staff found that the person himself/herself was sitting on the floor. The staff inputs "fall detected during night toilet assistance, the person himself/herself is conscious, vitals are stable, no external injury" from the terminal, and at the same time, the latest vital data from the wearable terminal is also automatically linked. A situation analysis unit comprehensively refers to the past fall history of the person himself/herself, the presence or absence of osteoporosis, the vital data, the intentions of the family, a care plan, the values of the person himself/herself of "want to avoid hospitalization", a night activity pattern, the latest rehabilitation record, and the like, and generates the following prompt for generative AI. Specifically, the system operates an edge cloud architecture in which a plurality of sensor nodes and a central server cooperate in this specific use case. The wearable terminal of the facility functions as an edge device including a 3-axis acceleration sensor and an optical heart rate sensor. The wearable terminal primarily processes raw data acquired at a sampling rate of 100 times per second on a local microcontroller, and extracts a feature vector. The wearable terminal continuously streams and transmits the extracted feature vector to the server via a low power wide area network. The situation analysis unit of the server buffers the received stream data in an in-memory database, and constantly executes an abnormality detection algorithm. The situation analysis unit detects a rapid change and a spatial displacement of acceleration data, and sets a fall event flag. For example, an input to the situation analysis unit includes an acceleration tensor and a time-series array of a heart rate from the wearable terminal, and text data of being conscious and having no external injury input from the terminal by the nursing care staff. The situation analysis unit outputs a context matrix in which a confirmation signal of a fall event and related personal data are integrated as an output. The situation analysis unit inputs the output context matrix to a natural language generation module, and dynamically constructs a prompt for inquiry to a large language model. The system integrates and processes a physical signal from a hardware sensor and semantic text information input by a human on the same vector space. Thereby, the system corrects a false detection of the sensor with a human input, and dramatically improves accuracy of situation recognition. The system exhibits a technical effect of minimizing latency from data collection to prompt generation, and enabling a quick initial response in an emergency. The system realizes advanced situation monitoring in specific application fields such as a smart nursing care facility and a fall prevention system of a hospital.

Prompt example: "Elderly woman with nursing care level 2, past illnesses are hypertension and osteoporosis, fall detected during night toilet assistance. The person herself has intentions to avoid hospitalization, top priority to pain relief, emphasis on visiting with family. History of two falls in the past, neither of which led to a serious matter. Current vitals are stable, no external injury. Family lives far away. For this fall, consider the values of the person herself and the intentions of the family, and explain an optimal response policy and a reason thereof in a natural language." Specifically, the system executes an encoding process of converting this prompt character string into a numerical representation processable by the large language model. The system maps each word and phrase in the prompt to an array of token IDs with reference to a pre-learned vocabulary dictionary. The system converts the token ID array into a continuous embedding vector in a high-dimensional space. The system further adds a positional encoding vector indicating position information in a sequence of each token. For example, an input to the system is the entire prompt character string described above. The system outputs a floating-point tensor having a sequence length and 1024 dimensions as an output. The system inputs the output tensor to a self-attention mechanism block of a transformer architecture. The system performs a process of mapping semantics of a natural language to a mathematical vector space, rather than simply passing a character string. Thereby, the system enables the large language model to quantitatively calculate a complex relationship between concepts such as nursing care level 2, hypertension, and hospitalization avoidance. The system brings about a technical effect of accurately transmitting an intention of the prompt to the inside of the model and maximizing accuracy of inference. The system functions as a foundation of a natural language interface in specific application fields such as medical text analysis and an automatic response system involving complex condition designation.

Upon receiving this prompt, the generative AI first evaluates a risk of a bone fracture or a head injury, and proposes "immediately contact the family and prioritize follow-up observation in the facility" when the vitals are stable and no external injury is recognized. When the person himself/herself complains of pain, it proposes "give top priority to pain relief, contact a doctor as necessary, and consider administration of an analgesic". If a vital abnormality or a head bruise is suspected, it proposes "recommend emergency transportation". The AI outputs a basis and a reason for each option in a natural language, such as "since the intentions to avoid hospitalization are registered as the wishes of the person himself/herself, recommend follow-up observation in the facility in cooperation with the family", "when the risk of a bone fracture or a head injury due to the fall is high, recommend prompt consultation at a medical institution", and "when the pain is severe, contact a doctor and consider administration of an analgesic". Specifically, the generative AI executes an inference operation using a multi-layer transformer decoder block based on the input prompt tensor. The generative AI calculates an attention weight between a current state of stable vitals and no external injury in the prompt and a values vector of hospitalization avoidance and pain relief through a self-attention mechanism. The generative AI outputs a probability distribution for a plurality of possible action candidates from a softmax layer based on the calculated weight. The generative AI selects an action candidate with the highest probability, and autoregressively generates word tokens one by one for explaining it. For example, an input to the generative AI is an embedding tensor of the prompt and an internal parameter of the model. The generative AI outputs a probability score of each action candidate and a token sequence of the generated natural language text as an output. The generative AI converts the output token sequence into a character string using a decoder, and transmits it to an interface unit. The generative AI executes an intuitive judgment of a human as a probabilistic optimization process in a high-dimensional space using billions of parameters. Thereby, the generative AI logically arbitrates conflicting conditions and derives an optimal solution. The generative AI exhibits a technical effect of generating a highly transparent output accompanied by a basis without making a process of decision-making a black box. The generative AI emulates a thinking process of an expert in specific application fields such as automatic generation of a clinical pathway at a medical site and decision-making support for a care policy involving an ethical dilemma.

Furthermore, when the family inputs a question such as "is hospitalization necessary for this fall?", "what is a recurrence prevention measure in the future?", or "how should a safety measure for a night toilet flow line be taken?" from the terminal, the generative AI returns a specific explanation such as "since no external injury is recognized and the vitals are stable in this fall, it is determined that hospitalization is unnecessary. As a recurrence prevention measure, strengthening lighting of the night toilet flow line, installing a fall prevention mat, utilizing a walking aid, strengthening a night monitoring system, and the like are effective." Specifically, the system processes the question text input from the terminal of the family via a dialogue management module. The dialogue management module combines a context vector regarding the immediately preceding fall event and an embedding vector of the new question text. The system inputs the combined vector to the large language model, and causes it to execute response generation maintaining a context. The large language model refers to an internal knowledge base and facility environment data acquired from a care receiver information management unit. For example, an input to the system includes a question text of how a safety measure for a night toilet flow line should be taken and an equipment information tensor of the facility. The system outputs structured data including a specific measure plan such as strengthening lighting and installing a fall prevention mat, and a text explaining it in a natural language as an output. The system transmits the output response text to the terminal of the family, and causes it to be displayed on a screen in a chat format. The system executes a multi-turn dialogue process linking a past event and a current situation inside a computer, rather than a single question answering. Thereby, the system saves the user the trouble of explaining a background of the situation every time, and realizes natural communication. The system brings about a technical effect of dramatically improving efficiency of information retrieval and reducing a cognitive load on the user. The system functions as an interactive information provision foundation in specific application fields such as a remote nursing care consultation system and a personalized healthcare assistant.

In addition, the system simulates various scenarios such as "night fall", "high fever occurrence", "aspiration during meal", "night wandering", "bedsore occurrence", and "medication error" by the generative AI, and enables virtual experience of a plurality of situations in advance, such as "flow when emergency transportation is selected at the time of night fall", "response when contacting a medical institution at the time of high fever occurrence", "first aid and contact to family when aspiration during meal occurs", "risk evaluation and additional measure when night wandering frequently occurs", "medical cooperation and care plan review when bedsore worsens", and "recurrence prevention measure when medication errors occur successively". Thereby, the person himself/herself, the family, and the nursing care staff can make sufficient preparation and consensus building in preparation for judgment in an emergency. Specifically, the system operates a distributed computing cluster for processing these various scenarios in parallel. A simulation unit defines each scenario as an independent calculation task, and allocates it to a plurality of calculation nodes. The calculation node sets an initial condition tensor in each scenario, and executes a state transition simulation based on a Markov decision process. The calculation node calls an inference API of the large language model at each time step, and calculates an optimal action policy under a virtual situation. The simulation unit aggregates calculation results from all nodes, and constructs a result tree for each scenario. For example, an input to the simulation unit includes a baseline health condition vector of the care receiver and a parameter of a virtual event of successive medication errors. The simulation unit outputs a probability distribution of an influence of a medication error on health and an effectiveness score of a recurrence prevention measure as an output. The simulation unit transmits the output result tree to the interface unit, and generates a dashboard allowing the user to interactively search the scenario. The system simulates decision-making of medical and nursing care, in which trial and error is not allowed in the real world, at high speed in a safe sandbox environment on a computer. Thereby, the system verifies responsiveness of the system to a black swan event having a low occurrence probability but a tremendous influence in advance. The system exhibits a technical effect of optimizing preparation for an unknown risk in a data-driven manner and enhancing robustness of the care plan. The system functions as an advanced prediction and analysis platform in specific application fields such as a training simulator for a medical worker and a stress test system for facility operation.

As described above, the system of the present invention manages various information of the care receiver in an integrated manner, analyzes a situation in real time using the generative AI, presents an optimal nursing care policy and care procedure, explains a basis and a reason thereof in a natural language, supports decision-making based on consent of the person himself/herself, the family, and the nursing care staff, and further enables advance preparation by scenario simulation and dynamic update of the care plan. Specifically, the system operates on a distributed computing environment. The system manages various information of the care receiver in an integrated manner as a multidimensional tensor. A data integration unit acquires a time-series array of vital signs, a natural language text of an electronic medical record, and a numerical vector of an environmental sensor. The data integration unit preprocesses the acquired data by a normalization process and a tokenization process. The system converts the preprocessed data into a 512-dimensional input vector. The system inputs the input vector to the large language model. The large language model adopts a transformer architecture having a self-attention mechanism. The large language model calculates a non-linear correlation of the input data in a high-dimensional feature space. This calculation process is a pure mathematical matrix operation different from human mental activity. The large language model receives, for example, a time-series array indicating a heart rate fluctuation for the past 72 hours as the input data. The large language model receives, for example, a text token sequence indicating a past fall history as the input data. The large language model receives, for example, a numerical vector indicating a current room temperature and humidity as the input data. The large language model infers an optimal nursing care policy based on these input data. The large language model outputs a probability distribution indicating a recommendation degree of each care procedure as an inference result. The large language model outputs an attention weight score serving as a basis as an inference result. The large language model outputs a token sequence of an explanatory text in a natural language as an inference result. The output data includes, for example, a probability value of 0.95 for a care procedure of immediate implementation of hydration. The output data includes, for example, a probability value of 0.05 for a care procedure of emergency transportation. The output data includes, for example, a contribution score of 0.88 for an abnormal heart rate value. The system executes a threshold determination process on the output probability distribution. The system extracts a care procedure having a probability value exceeding a predetermined threshold. The system dynamically updates a care plan database using the extracted care procedure. The system generates a basis explanatory text in a natural language based on the attention weight score. The system transmits the generated explanatory text to a display module of the terminal. The system includes a scenario simulation unit. The scenario simulation unit generates a plurality of virtual future state vectors using a Monte Carlo method. The scenario simulation unit re-inputs the generated future state vectors to the large language model. The system predicts a future fluctuation of the care plan by this re-input process. The system renders a prediction result as a time-series transition graph. The system significantly reduces latency of data processing by these processes. The system executes multivariate analysis exceeding a human cognitive limit. Thereby, the system dramatically improves determination accuracy of the nursing care policy. The large language model is pre-learned in advance using a large-scale medical and nursing care corpus. This learning process updates a weight parameter of a network so as to minimize a cross-entropy error function. This learning process uses a gradient descent algorithm. The system realizes improvement of computer technology itself, rather than mere business efficiency improvement, by these technical configurations. The system adopts a hybrid architecture combining a specific rule-based process and a machine learning model. Thereby, the system prevents a black box and ensures transparency of inference. The system exhibits an extremely high technical effect in a specific use case of real-time decision-making support at a medical site or a nursing care facility.

The specific processing unit 290 sends the results of specific processing obtained in Example 1 or Application Example 1 described above to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence).

An example of the data generation model 58 is a generative AI such as ChatGPT (registered trademark) (Internet search <URL: https://openai.com/blog/chatgpt>).

The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

In the above embodiments, an example form where specific processing is performed by the data processing device 12 was described, but the technology disclosed herein is not limited to this, and specific processing may be performed by the smart device 14.

### Second Embodiment

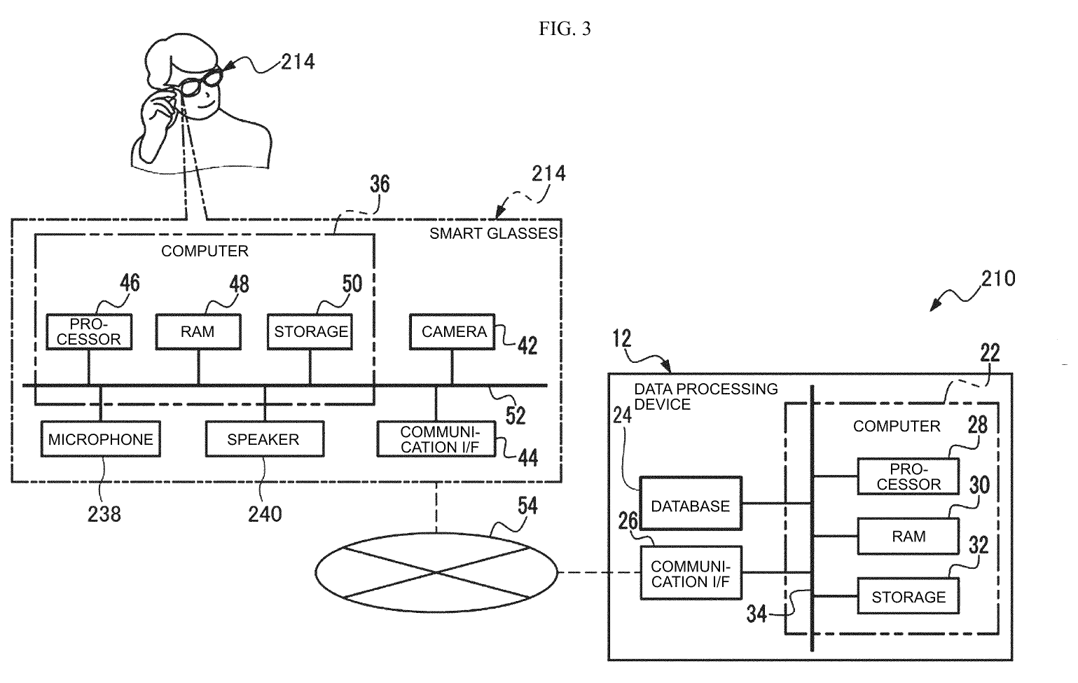

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 is an example of a "computer" related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, camera 42, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

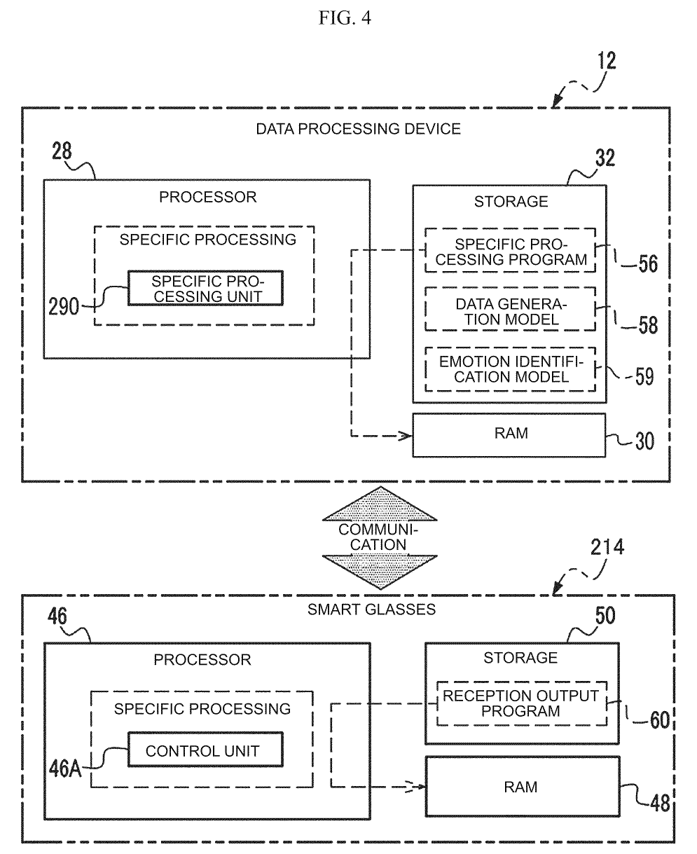

FIG. 4 shows an example of the main functions of the data processing device 12 and smart glasses 214. As shown in FIG. 4, specific processing is performed in the data processing device 12 by the processor 28. The storage 32 stores a specific processing program 56.

The specific processing program 56 is an example of a "program" related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, and various estimations and predictions related to the user's emotions are performed, but it is not limited to such examples. Furthermore, emotion estimation and prediction may include, for example, emotion analysis.

In the smart glasses 214, reception output processing is performed by the processor 46. The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48. The smart glasses 214 may also have similar data generation models and emotion identification models as the data generation model 58 and emotion identification model 59, and perform the same processing as the specific processing unit 290 using these models.

Next, specific processing by the specific processing unit 290 of the data processing device 12 will be described. Each part of the system described below is realized by the data processing device 12 and the smart glasses 214. In the following description, the data processing device 12 is referred to as a "server," and the smart glasses 214 are referred to as a "terminal."

(Example 1)

Since the flow of the specific processing is the same as that in Example 1 described in the first embodiment, the description thereof is omitted.

(Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

In the above embodiments, an example form where specific processing is performed by the data processing device 12 was described, but the technology disclosed herein is not limited to this, and specific processing may be performed by the smart glasses 214.

### Third Embodiment

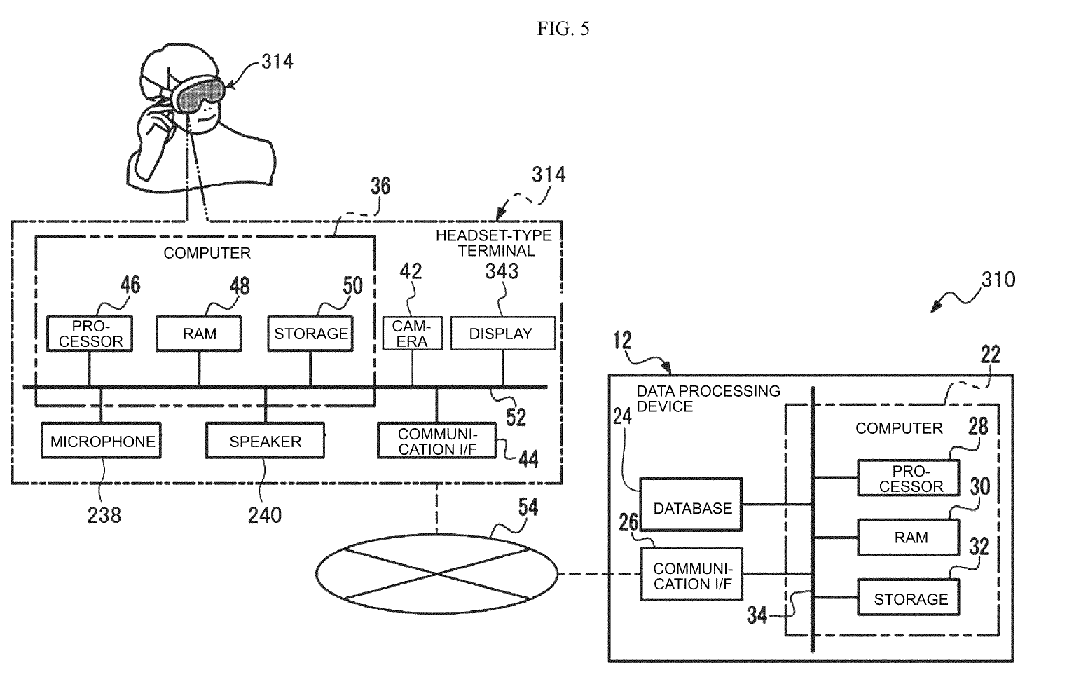

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 is an example of a "computer" related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, display 343, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

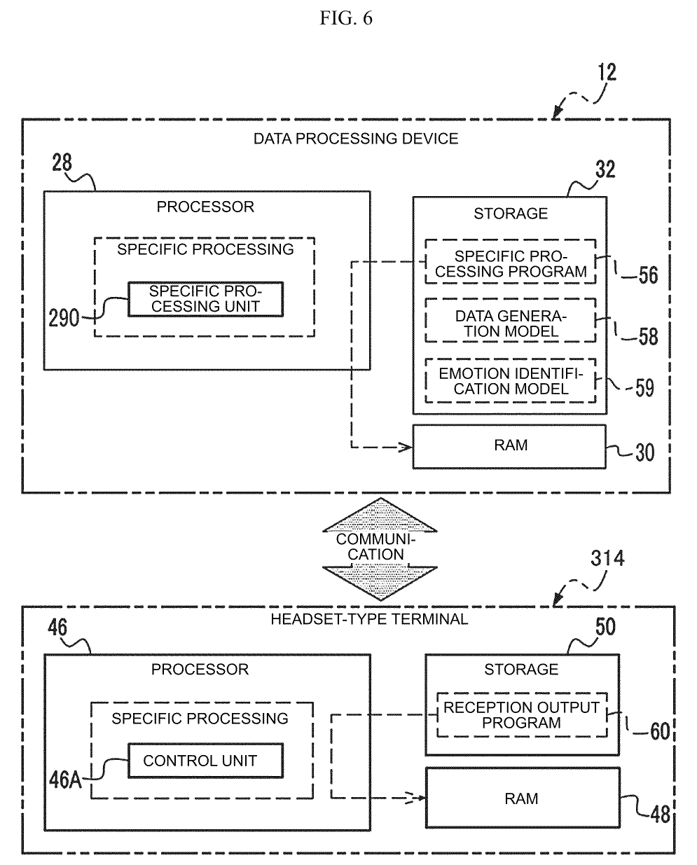

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The specific processing program 56 is an example of a "program" related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290.

In the headset-type terminal 314, reception output processing is performed by the processor 46.

The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48.

Next, specific processing by the specific processing unit 290 of the data processing device 12 will be described. Each part of the system described below is realized by the data processing device 12 and the headset-type terminal 314. In the following description, the data processing device 12 is referred to as a "server," and the headset-type terminal 314 is referred to as a "terminal."

(Example 1) Since the flow of the specific processing is the same as that in Example 1 described in the first embodiment, the description thereof is omitted.

(Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

In the above embodiments, an example form where specific processing is performed by the data processing device 12 was described, but the technology disclosed herein is not limited to this, and specific processing may be performed by the headset-type terminal 314.

### Fourth Embodiment

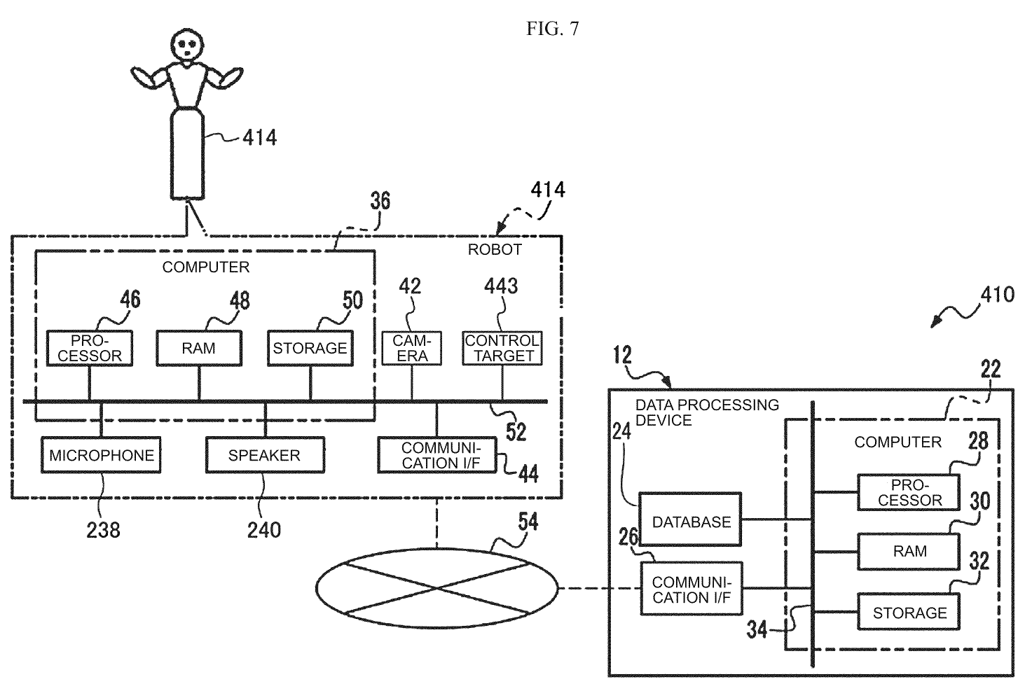

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 is an example of a "computer" related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, control target 443, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

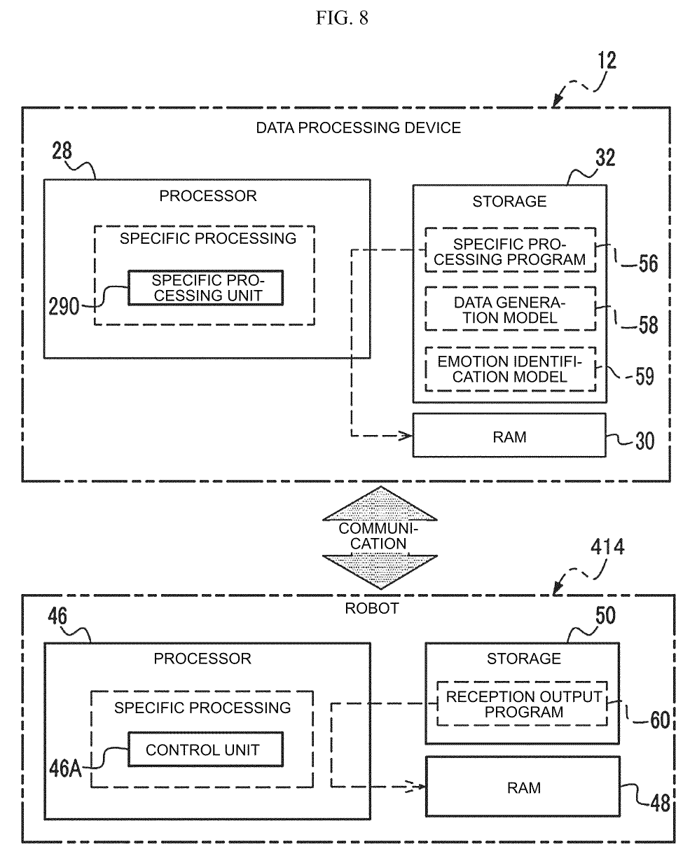

FIG. 8 shows an example of the main functions of the data processing device 12 and the robot 414. As shown in FIG. 8, specific processing is performed in the data processing device 12 by the processor 28. The storage 32 stores a specific processing program 56.

The specific processing program 56 is an example of a "program" related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290.

In the robot 414, reception output processing is performed by the processor 46. The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48.

Next, specific processing by the specific processing unit 290 of the data processing device 12 will be described. Each part of the system described below is realized by the data processing device 12 and the robot 414. In the following description, the data processing device 12 is referred to as a "server," and the robot 414 is referred to as a "terminal."

(Example 1) Since the flow of the specific processing is the same as that in Example 1 described in the first embodiment, the description thereof is omitted.

(Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

In the above embodiments, an example form where specific processing is performed by the data processing device 12 was described, but the technology disclosed herein is not limited to this, and specific processing may be performed by the robot 414.

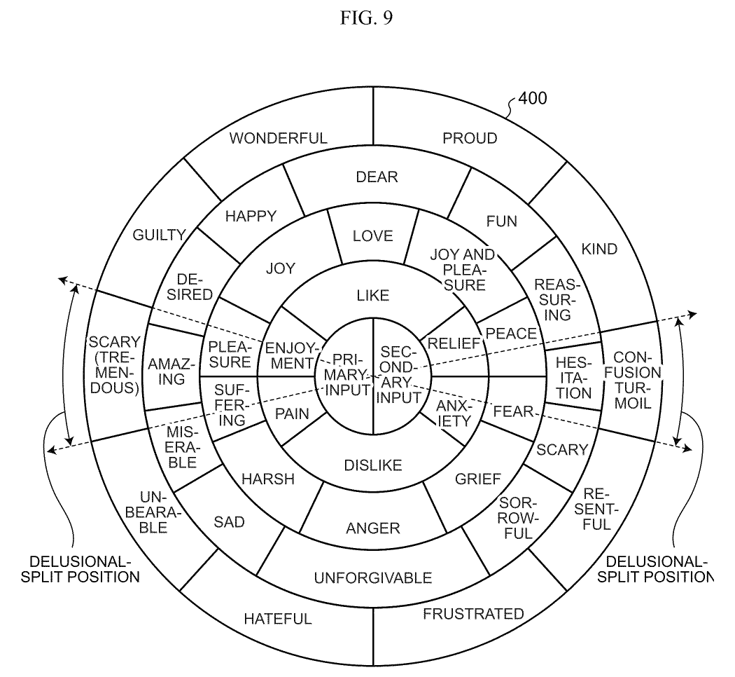

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, "pleasant" emotions are arranged, and on the lower side, "unpleasant" emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called "reactions," where sensations take precedence, are aligned. Additionally, in the right half of the emotion map, emotions belonging to the domain called "situations," where situational recognition takes precedence, are aligned.

In the emotion map, two emotions that promote learning are defined. One is a negative emotion around "repentance" or "reflection" on the situation side. In other words, when a negative emotion arises in the robot, like "I never want to feel this way again" or "I don't want to be scolded again." The other is an emotion around "desire" on the reaction side, which is positive. In other words, it is a positive feeling like "I want more" or "I want to know more."

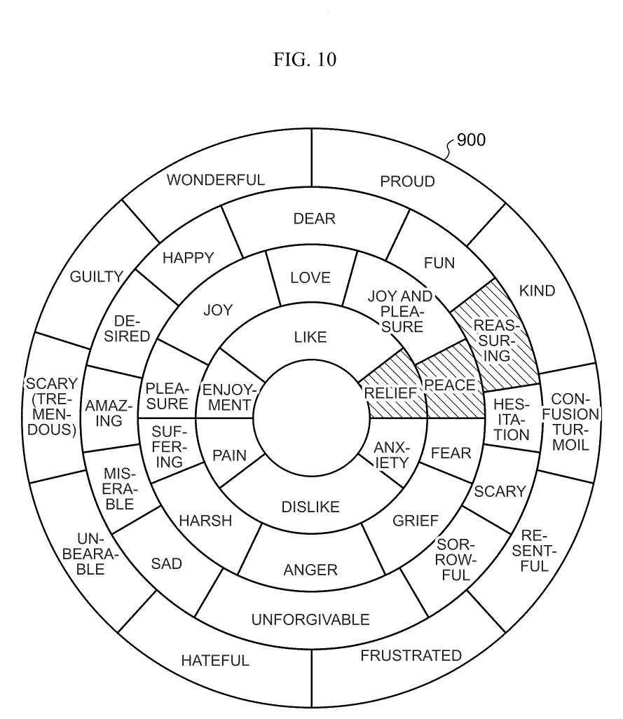

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like "reassured," "calm," and "confident" have similar emotion values.

In the above, the system according to the present disclosure has been described mainly on the functions of the data processing device 12, but the system according to the present disclosure is not necessarily mounted on a server. The system according to the present disclosure may be implemented as a general information processing system. The present disclosure may be implemented, for example, as a software program running on a personal computer, or an application running on a smartphone or the like. The method according to the present disclosure may be provided to the user in the form of SaaS (Software as a Service).

In the above embodiments, an example form where specific processing is performed by a single computer 22 was described, but the technology disclosed herein is not limited to this, and distributed processing for specific processing by multiple computers including the computer 22 may be performed. For example, the data generation model 58 may be provided in an external device of the data processing device 12, and the external device may generate data corresponding to the input data.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned.

Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### Note that the following supplementary notes are further disclosed regarding the above description.

### Supplementary Note 1

A system comprising: a care receiver information management unit; a situation analysis unit; and a nursing care decision-making support unit, wherein the care receiver information management unit has a function of acquiring various personal data such as a medical history, a nursing care history, lifestyle habits, values, intentions of a family, legal information, a care plan, an advance directive, vital data, behavior data from a wearable terminal, and input information by a nursing care staff or a family of a care receiver himself/herself from an external service or a terminal at a nursing care site, managing these in an integrated and dynamic manner, and updating these in real time according to a change in a situation and a new input; the situation analysis unit is configured to analyze situation data such as a fall, a fever, a vital abnormality, a behavior change, a nursing care record, and a report from a family or a staff of the care receiver in an emergency and a normal time using generative AI, and determine a degree of urgency and a necessary response; and the nursing care decision-making support unit is configured to comprehensively consider values and wishes of the person himself/herself, the intentions of the family, the care plan, a situation of a medical site, social ethical codes, and the like based on information obtained from the care receiver information management unit and the situation analysis unit, propose an optimal nursing care policy and care procedure using an ethical decision-making algorithm by the generative AI, further explain a basis and a reason for the option with high transparency in a natural language, and support decision-making based on understanding and consent of the nursing care staff and the family.

### Supplementary Note 2

The system according to Supplementary Note 1, wherein the nursing care decision-making support unit is configured to explain complex options, care procedures, and contents of legal procedures in an emergency such as a fall or a fever and a daily care judgment to the care receiver himself/herself, the family, and the nursing care staff in an easy-to-understand manner via a natural language dialogue interface by the generative AI, enable the person himself/herself, the family, and the nursing care staff to input their own intentions and wishes in a dialogue format, dynamically update the advance directive and the care plan by immediately reflecting the input contents in the care receiver information management unit, and propose an optimal option again by the generative AI when the situation analysis unit detects a new situation change.

### Supplementary Note 3

The system according to Supplementary Note 1, wherein the nursing care decision-making support unit is configured to virtually simulate various emergencies and nursing care and medical scenarios by the generative AI, present a result of decision-making based on the values and wishes of the person himself/herself and the care plan and an influence thereof in a natural language for each scenario to the care receiver himself/herself, the family, and the nursing care staff, enable the person himself/herself, the family, and the nursing care staff to prepare and review decision-making while experiencing a plurality of situations in advance, and automatically update the advance directive, the personal information, and the care plan based on a simulation result.

What is claimed is:

1. A system comprising:
circuitry configured to:
receive, via a communication interface coupled to a packet-switched network, subject profile data records and contextual state data from a terminal device, the subject profile data records comprising historical record data, preference parameter records, and directive data, and store the subject profile data records in a storage device;
monitor streaming state data received from the terminal device and external data services via the communication interface, apply a natural language processing model to unstructured text data in the contextual state data to extract urgency classification labels and situational feature vectors, and update subject profile data records in real time based on detected state changes;
apply a generative neural network model to the subject profile data records, the urgency classification labels, and the situational feature vectors to generate a ranked option data record comprising a plurality of candidate response sequences with associated justification text, the candidate response sequences generated with reference to preference parameter records and directive data retrieved from the storage device;
apply an ethical constraint evaluation model to filter the ranked option data record by computing compatibility scores between each candidate response sequence and preference parameter records, and retain candidate response sequences having compatibility scores exceeding a compatibility threshold; and
transmit the filtered ranked option data record and the associated justification text to terminal devices via the communication interface.

2. The system according to claim 1, wherein the circuitry is configured to
receive updated directive data from a terminal device via the communication interface comprising preference updates and constraint parameter updates, apply the natural language processing model to the updated directive data to extract structured preference parameters, and update the corresponding subject profile data records in the storage device in real time.

3. The system according to claim 2, wherein the circuitry is configured to
apply an ambiguity classification model to the updated directive data to generate an ambiguity score, generate a clarification prompt comprising targeted clarification questions when the ambiguity score exceeds an ambiguity threshold, and transmit the clarification prompt to the terminal device prior to updating the subject profile data record.

4. The system according to claim 1, wherein the circuitry is configured to
apply a simulation model to each candidate response sequence to generate a projected outcome trajectory comprising a set of state transition probability values, and incorporate the projected outcome trajectory as a simulation result field in the ranked option data record for presentation to the terminal device.

5. The system according to claim 4, wherein the circuitry is configured to
generate the projected outcome trajectory by applying the generative neural network model to the candidate response sequence, historical outcome records retrieved from a vector database by cosine similarity search, and contextual state data, and computing a weighted probability distribution over possible future state transitions.

6. The system according to claim 1, wherein the circuitry is configured to
apply a multi-party preference aggregation algorithm to preference parameter records associated with a plurality of authorized contact user identifiers for a subject identifier, compute a consensus preference vector by weighted aggregation of individual preference parameter records, and incorporate the consensus preference vector into the ranked option data record generation process.

7. The system according to claim 1, wherein the circuitry is configured to
receive streaming sensor data from wearable monitoring devices associated with the subject identifier via the communication interface, apply an anomaly detection model to the streaming sensor data to compute reconstruction error scores, and update the urgency classification label when a reconstruction error score exceeds an urgency reclassification threshold.

8. The system according to claim 7, wherein the circuitry is configured to
generate an escalated alert command when the urgency classification label transitions to a high-urgency category, and transmit the escalated alert command to terminal devices associated with designated contact user identifiers and external emergency response service endpoints via the communication interface.

9. The system according to claim 1, wherein the circuitry is configured to
generate justification text for each candidate response sequence by applying the generative neural network model to the candidate response sequence, the associated compatibility score, and relevant preference parameter records, producing a natural language explanation of the basis and rationale for the candidate response sequence for transmission to the terminal device.

10. The system according to claim 9, wherein the circuitry is configured to
receive a selection input from the terminal device identifying a selected candidate response sequence, store the selection input with the associated justification text as a decision record in the storage device, and update the subject profile data record with a consent confirmation field derived from the selection input.

11. The system according to claim 1, wherein the circuitry is configured to
store all state data updates, ranked option data records, selection inputs, and consent confirmation records in an audit log data structure in the storage device with timestamps and user identifiers, and maintain the audit log data structure as a tamper-resistant append-only log.

12. The system according to claim 1, wherein the circuitry is configured to
apply the natural language processing model to historical decision records stored in the storage device to extract preference pattern features, update the preference parameter records by incorporating the extracted preference pattern features, and regenerate the ranked option data record using the updated preference parameter records.

13. The system according to claim 1, wherein the circuitry is configured to
apply a dialogue management model to enable iterative natural language interaction with a terminal device user regarding each candidate response sequence, generate follow-up explanation text in response to clarification queries received via the communication interface, and incorporate user responses into an updated ranked option data record.

14. The system according to claim 1, wherein the circuitry is configured to
deploy each functional unit as an independent containerized microservice, execute data transfer between containerized microservices via a message queue using a publish-subscribe model, and scale each microservice independently based on load metrics to maintain system availability.

15. The system according to claim 1, wherein the circuitry is configured to
enforce an access control policy by verifying a user role parameter associated with the terminal device against an authorized role set before permitting access to subject profile data records, and apply data minimization processing to return only the data fields authorized for the requesting user role parameter.

16. The system according to claim 1, wherein the circuitry is configured to
update the generative neural network model by generating training pairs from historical ranked option data records and corresponding validated selection inputs, and fine-tune the generative neural network model on the training pairs to improve compatibility score prediction accuracy.

17. The system according to claim 1, wherein the subject profile data records comprise historical state records, contextual preference parameters, and structured directive data representing expressed preferences for future state scenarios, and the ranked option data record presents candidate response sequences ordered by compatibility with the subject's expressed preferences.

18. A system comprising:
circuitry configured to:
receive, via a communication interface coupled to a packet-switched network, subject profile data records comprising historical record data, preference parameter records, and directive data, and contextual state data from terminal devices and external data services;
apply a natural language processing model to unstructured text data to extract urgency classification labels and situational feature vectors, and update subject profile data records in real time based on detected state changes;
apply a generative neural network model to the subject profile data records, urgency classification labels, and situational feature vectors to generate a ranked option data record, and apply an ethical constraint evaluation model to filter candidate response sequences by compatibility scores with preference parameter records;
generate justification text for each retained candidate response sequence using the generative neural network model; and
transmit the filtered ranked option data record and justification text to terminal devices, and receive selection inputs identifying selected candidate response sequences for storage as decision records.

19. The system according to claim 18, wherein the circuitry is configured to
apply a simulation model to each candidate response sequence to generate a projected outcome trajectory comprising state transition probability values, apply a multi-party preference aggregation algorithm to compute a consensus preference vector from preference parameter records of a plurality of authorized contact user identifiers, and incorporate both the projected outcome trajectory and the consensus preference vector into the ranked option data record.

20. A method comprising:
receiving, via a communication interface coupled to a packet-switched network, subject profile data records and contextual state data from a terminal device, the subject profile data records comprising historical record data, preference parameter records, and directive data, and storing the subject profile data records in a storage device;
monitoring streaming state data, applying a natural language processing model to extract urgency classification labels and situational feature vectors, and updating subject profile data records in real time based on detected state changes;
applying a generative neural network model to the subject profile data records, the urgency classification labels, and the situational feature vectors to generate a ranked option data record comprising candidate response sequences with associated justification text;
applying an ethical constraint evaluation model to filter the ranked option data record by compatibility scores with preference parameter records; and
transmitting the filtered ranked option data record and the associated justification text to terminal devices via the communication interface.
